(19) United States
(12) Patent Application Publication
(10) Pub. No.: US 2024/0090959 A1
(43) Pub. Date: Mar. 21, 2024

(54) DEVICES AND METHODS FOR FORCE SENSING UNIT WITH SHAFT TRANSLATION AND ROLL

(71) Applicant: Intuitive Surgical Operations, Inc., Sunnyvale, CA (US)

(72) Inventor: Rumen DEYANOV, Fresno, CA (US)

(73) Assignee: Intuitive Surgical Operations, Inc., Sunnyvale, CA (US)

(21) Appl. No.: 18/266,141

(22) PCT Filed: Dec. 15, 2021
(86) PCT No.: PCT/US2021/063490

§ 371 Date: Jun. 8, 2023

Related U.S. Application Data
(60) Provisional application No. 63/126,770, filed on Dec. 17, 2020.

(51) Int. Cl. A61B 34/30; A61B 34/00

(52) U.S. Cl. CPC A61B 34/30; A61B 34/76; A61B 2034/301; A61B 2034/305

(57) ABSTRACT

A surgical instrument includes a support structure, a shaft, a shaft translation carriage including a shaft roll carrier, a shaft roll drive group, and a force sensor unit. The shaft comprises a proximal end and a distal end, and a shaft axis is defined by the proximal and distal ends. The shaft is coupled to the support structure by the shaft roll carrier. The shaft roll drive group is configured to rotate the shaft about the shaft axis and comprises a shaft roll driver, a shaft roll drive receiver, and a shaft roll drive coupling. The shaft roll drive receiver translates along the shaft axis relative to the shaft roll driver as the shaft translates along the shaft axis. The force sensor unit is configured to produce a the shaft axis.

### RELATED APPLICATIONS

This patent application claims priority to and the filing date benefit of U.S. Provisional Patent Application No. 63/126,770 (filed Dec. 17, 2020) (entitled “DEVICES AND METHODS FOR FORCE SENSING UNIT WITH SHAFT TRANSLATION AND ROLL”), which is incorporated by reference herein in its entirety.

This application is related to U.S. Provisional Patent Application No. 63/077,833 (filed Sep. 14, 2020) (entitled “DEVICES AND METHODS FOR COMPACT, REDUNDANT INDUCTIVE FORCE SENSOR”), which is incorporated by reference herein in its entirety.

This application is also related to U.S. Provisional Patent Application No. 62/901,729 (filed Sep. 17, 2019) (entitled “COMPACT, DIFFERENTIAL, COAXIAL INDUCTIVE FORCE SENSOR”) and to PCT International Patent Application No. PCT/US2020/050696 (filed Sep. 14, 2020) (entitled “COMPACT, DIFFERENTIAL, COAXIAL INDUCTIVE FORCE SENSOR”) which are incorporated herein by reference in their entirety.

### BACKGROUND

The embodiments described herein relate to force sensing mechanical structures, more specifically to medical devices that incorporate force sensing mechanical structures, and still more specifically to instruments used for minimally invasive surgery and that incorporate force sensing mechanical structures. More particularly, the embodiments described herein relate to medical devices that include a force sensor unit that is coupled to a mechanical structure of the medical device and is used to measure axial forces applied to the end effector of the medical device during a surgical procedure. The medical devices described herein also provide for measuring axial forces while also allowing translational and rotational movement of a shaft of a medical device.

Known techniques for minimally invasive medical interventions employ instruments that can be either manually controlled or controlled via hand-held or mechanically grounded teleoperated medical systems that operate with at least partial computer-assistance (“telesurgical systems”) so as to therapeutic and diagnostic functions on patient tissue. Many known medical instruments include a therapeutic or diagnostic end effector (e.g., forceps, a cutting tool, a cauterizing tool, or an imaging device) mounted on an optional wrist mechanism at the distal end of a shaft. During a medical procedure, the end effector, wrist mechanism, and the distal end of the shaft are inserted into a small incision or a natural orifice of a patient to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector, and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the end effector's roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

To enable the desired movement of the wrist mechanism and end effector, known instruments include mechanical connectors (e.g., cables) that extend through the shaft of the instrument and that connect the distal wrist mechanism to a proximal mechanical structure used to move the connectors to operate the wrist mechanism. For telesurgical systems, the mechanical structure is typically motor driven and operably coupled to a computer processing system to provide a user interface for a clinical user (e.g., a surgeon) to control the instrument as a whole and the instrument's components and functions.

Force sensing surgical instruments are known and together with associated telesurgical systems produce associated haptic feedback to a clinical user during a medical procedure, which brings better user immersion, realism, and intuitiveness (i.e., more effective telepresence) to a clinician performing the procedure. For effective haptics rendering and accuracy, force sensors are placed on a medical instrument. One approach is to include a force sensor unit attached to and/or incorporated within the proximal mechanical structure of the medical instrument and that can be used to measure axial forces imparted on the end effector of the medical instrument. These force measurements are measured at or near the instrument shaft and are used to produce haptic feedback forces at an input to a master control device to provide to a user an indication of the forces imparted by the medical instrument to, for example, patient tissue. That is, a force imparted by an instrument on objects such as tissue or suture are indicated by a corresponding reactive force from such objects on the instrument, and the sensed reactive force is conveyed to the user as a haptic sensation.

Enhancements to force sensor systems lead to more accurate force measurements, which in turn, result in more accurate haptic feedback. For example, including multiple sensors to measure a single force parameter (e.g., the axial force imparted on the end effector) can improve measurement accuracy (e.g., by producing an average measurement or by allowing for subtraction of commons modes) and allow for operation if one sensor fails. The inclusion of additional sensors, however, competes for the limited space that exists because of the mechanical structure and overall instrument size restrictions required by minimally invasive medical instruments. Force sensor systems must not only be as effective as possible, they must fit within the spatial design constrains of objects experiencing the force, such as medical instruments. Additionally, force sensor systems for measuring the axial force imparted on the end effector must also be able to accommodate translation (e.g., along the shaft axis) and rotation (e.g., a roll DOF) of the shaft relative to the mechanical structure.

Thus, a need exists for improved force-sensing capabilities that can in turn improve haptic feedback, especially within the spatial constraints of minimally invasive surgical instruments. There is also a need for improvements in providing translational and rotational movement of the shaft of a medical device.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter. In some embodiments, a surgical instrument includes a support structure, a shaft, a shaft translation carriage, a shaft roll carriage, a shaft roll drive group, and a force sensor unit. The shaft translation carriage can include the shaft roll carriage. The shaft comprises a proximal end and a distal end, and a shaft axis is defined by the proximal and distal ends. The shaft is coupled to the support structure by the shaft roll carriage. The shaft roll drive group is configured to rotate the shaft about the shaft axis and comprises a shaft roll driver coupled to the support structure, a shaft roll drive receiver coupled to the shaft, and a shaft roll drive coupling coupled between the shaft roll driver and the shaft roll drive receiver. The shaft roll drive receiver translates along the shaft axis relative to the shaft roll driver as the shaft translates along the shaft axis. The force sensor unit is configured to produce a signal associated with an amount of a force imparted to the shaft along the shaft axis.

In some embodiments, the shaft translation carriage is structured to constrain shaft translation relative to the support structure to translation along the shaft axis and the shaft roll carriage is structured to constrain shaft roll relative to the support structure to roll about the shaft axis. In some embodiments, the shaft translation carriage comprises a resiliency that urges the shaft to a defined lowest energy location along the shaft axis. In some such embodiments, the resiliency comprises one or more springs coupled between the support structure and the shaft translation carriage. In some such embodiments, the resiliency is inherent in the shaft translation carriage.

In some embodiments, the shaft translation carriage comprises a spring, and the spring is configured to be displaced in proportion to a force imparted to the shaft in a direction along the shaft axis. In some embodiments, the force sensor unit further comprises a sensor and a signal generated by the sensor is associated with a linear displacement of the shaft as the shaft translates along the shaft axis.

In some embodiments, the force sensor unit comprises an inductive sensor and a microprocessor, the inductive sensor is configured to generate a signal associated with a position of the shaft as the shaft translates along the shaft axis, and the microprocessor is configured to receive the signal. In some embodiments, the shaft roll drive coupling comprises a cable.

In some embodiments, a surgical instrument comprises a shaft comprising a proximal end and a distal end, and a shaft axis defined by the proximal and distal ends. The surgical instrument further comprises means for constraining the shaft to translation along the shaft axis in response to a force applied at the distal end of the shaft, means for driving the shaft to rotate about the shaft axis as the shaft is displaced in translation along the shaft axis, and means for determining an amount of the force applied at the distal end of the shaft along the shaft axis.

In some embodiments, the means for determining an amount of the force comprises means for sensing an amount of displacement of the shaft along the shaft axis. In some embodiments, the surgical instrument further comprises means for urging the shaft to a lowest energy location along the shaft axis. In some embodiments, the surgical instrument further comprises means for providing a resiliency to translation of the shaft along the shaft axis.

In some embodiments, the surgical instrument further comprises a support structure and the shaft is coupled to the base. The means for providing a resiliency to the shaft comprises one or more springs coupled between the shaft and the support structure. In some embodiments, the surgical instrument further comprises a shaft translation carriage and the shaft is coupled to the shaft translation carriage. The means for constraining the shaft to translation along the shaft axis in response to the force applied at the distal end of the shaft comprises the shaft translation carriage, and the means for providing a resiliency to translation of the shaft along the shaft axis is inherent in the shaft translation carriage.

In some embodiments, the means for driving the shaft to rotate about the shaft axis as the shaft is displaced in translation along the shaft axis comprises a shaft roll driver, a shaft roll drive receiver coupled to the shaft, and a shaft roll drive coupling coupled between the shaft roll driver and the shaft roll drive receiver. In some embodiments, the shaft roll drive coupling comprises a cable.

In some embodiments the surgical instrument further comprises means for generating a signal associated with a linear displacement of the shaft as the shaft translates along the shaft axis. In some embodiments, the means for driving the shaft to rotate comprises a cable.

In some embodiments a surgical instrument comprises a mechanical structure, a shaft comprising a proximal end portion and a distal end portion, a force sensor unit, a shaft roll drive receiver coupled to the proximal end portion of the shaft, and a shaft roll driver coupled to the shaft roll drive receiver. A shaft axis extends between the proximal and distal end portions of the shaft and the force sensor unit is configured to produce a signal associated with a force imparted to the shaft in a direction along the shaft axis. The shaft roll drive receiver and the shaft translate along the shaft axis and the shaft roll driver rotates the shaft roll drive receiver, and the shaft roll drive receiver rotates the shaft about the shaft axis.

In some embodiments, the shaft roll driver comprises a shaft roll drive coupling coupled to the shaft roll drive receiver, and the shaft roll drive coupling comprises a cable. In some embodiments, the surgical instrument further comprises a shaft roll carriage coupled to the mechanical structure and to the shaft, the shaft roll drive carrier is movable with the shaft along the shaft axis, and the shaft roll drive carrier remains stationary as the shaft rotates about the shaft axis.

In some embodiments, the surgical instrument further comprises a shaft translation carriage, and the shaft translation carriage comprises a spring. The spring is configured to be displaced in proportion to the force imparted to the shaft in the direction along the shaft axis. In some embodiments, the force sensor unit further comprises a sensor; and a signal generated by the sensor is associated with a linear displacement of the shaft as the shaft translates along the shaft axis. In some embodiments, the force sensor unit comprises an inductive sensor and a microprocessor communicatively coupled to the inductive sensor, the inductive sensor is configured to generate a signal associated with a position of the shaft as the shaft moves along the shaft axis, and the microprocessor receives the signal.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control unit of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is a diagrammatic illustration of a medical device including a force sensor unit, according to an embodiment.

FIG. 6A is a diagrammatic illustration of a portion of a medical device, according to an embodiment, shown in a first configuration.

FIG. 6B is a diagrammatic illustration of a portion of a medical device, according to an embodiment, shown in a second configuration.

FIG. 6C is a diagrammatic illustration of a portion of a medical device, according to an embodiment.

FIG. 7 is a perspective view of a medical device, according to an embodiment.

FIG. 8 is an enlarged perspective view of a distal end portion of the medical device of FIG. 7A.

FIG. 9 is a top view of the mechanical structure of the medical device of FIG. 7.

FIG. 10 is a perspective view of a portion of the medical device of FIG. 7.

FIG. 11 is a perspective view of a portion of the medical device of FIG. 7 with select components removed for illustration purposes.

FIG. 12 is a side view of a portion of the medical device of FIG. 7 with select components removed for illustration purposes.

FIG. 13 is a perspective view of a shaft translation carriage of the medical device of FIG. 7.

FIG. 14 is a perspective view of a flexure a force sensor unit of the medical device of FIG. 7.

FIG. 15 is a side view of a portion of the medical device of FIG. 7 with select components removed and showing a roll receiver and roll driver of the medical device.

FIG. 16 is a side view of the roll receiver, roll driver and shaft of FIG. 15.

FIG. 17 is a side view of a portion of a medical device according to an embodiment.

FIG. 18 is a perspective view of the mechanical structure of the medical device of FIG. 17 with select components removed for illustration purposes.

FIG. 19 is a side view of the mechanical structure and shaft of the medical device of FIG. 17 with select components removed for illustration purposes.

FIG. 20 is a side view of a linkage and shaft of the medical device of FIG. 17 with select components removed for illustration purposes.

FIG. 21 is a partially exploded view of the linkage and shaft of FIG. 20.

FIGS. 22 and 23 are each a different perspective views of the mechanical structure of the medical device of FIG. 17 with select components removed for illustration purposes.

FIG. 24 is a perspective view of a link of a force sensor unit of the medical device of FIG. 17.

FIG. 25 is a side view of the mechanical structure of the medical device of FIG. 17 with select components removed for illustration purposes and showing the spring and shaft in a first neutral position.

FIGS. 26 and 27 are each a side view of the mechanical structure of the medical device of FIG. 17 with select components removed for illustration purposes and showing the shaft in a second upper position (FIG. 26) and a third lower position (FIG. 27).

FIG. 28 is a perspective view of the shaft coupled to the roll receiver and roll carrier of the medical device of FIG. 17.

FIG. 29 is an exploded perspective view of the shaft coupled to the roll receiver and roll carrier of the medical device of FIG. 28.

FIG. 30 is a side view of the mechanical structure of the medical device of FIG. 17 with select components removed for illustration purposes and showing the shaft coupled to the roll receiver.

FIG. 31 is a side view of the coil assembly of the force sensor unit of the medical device of FIG. 17.

FIG. 32 is a diagrammatic illustration of a portion of a force sensor unit, according to an embodiment.

### DETAILED DESCRIPTION

The embodiments described herein can advantageously be used in a wide variety of force sensor applications, such as for grasping, cutting, and manipulating operations associated with minimally invasive surgery. The embodiments described herein can also be used in a variety of non-medical applications such as, for example, teleoperated systems for search and rescue, remotely controlled submersible devices, aerial devices, and automobiles, etc. The medical instruments or devices of the present application enable motion in three or more mechanical degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF). Thus, in some embodiments, the medical instruments or devices of the present application enable motion in six DOFs. The embodiments described herein further can be used to determine the forces exerted on (or by) a distal end portion of the instrument during use.

In some embodiments, the medical instruments described herein include a support structure, a shaft, a shaft translation carriage, a shaft roll carriage, a shaft roll drive group, and a force sensor unit. The shaft roll drive group is configured to rotate the shaft about a shaft axis and comprises a shaft roll driver coupled to the support structure, a shaft roll drive receiver coupled to the shaft, and a shaft roll drive coupling coupled between the shaft roll driver and the shaft roll drive receiver. The shaft roll drive receiver translates along the shaft axis relative to the shaft roll driver as the shaft translates along the shaft axis. The shaft translation carriage also translates along the shaft axis and is structured to constrain shaft translation relative to the support structure to translation along the shaft axis. The shaft roll driver is configured to cause the shaft roll drive receiver and the shaft to rotate relative to the support structure, but the shaft translation carriage does not rotate with the shaft roll drive receiver and the shaft. The force sensor unit is configured to produce a signal associated with an amount of a force imparted to the shaft along the shaft axis.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial device positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms “apparatus”, “medical device”, “instrument”, and variants thereof, can be interchangeably used.

Aspects of the invention are described primarily in terms of an implementation using a da Vinci® Surgical System, commercialized by Intuitive Surgical, Inc. of Sunnyvale, California. Examples of such surgical systems are the da Vinci Xi® Surgical System (Model IS4000), and the da Vinci X® Surgical System (Model IS4200). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® Surgical Systems (e.g., the Model IS4000, the Model IS4200) are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support from a mechanical ground.

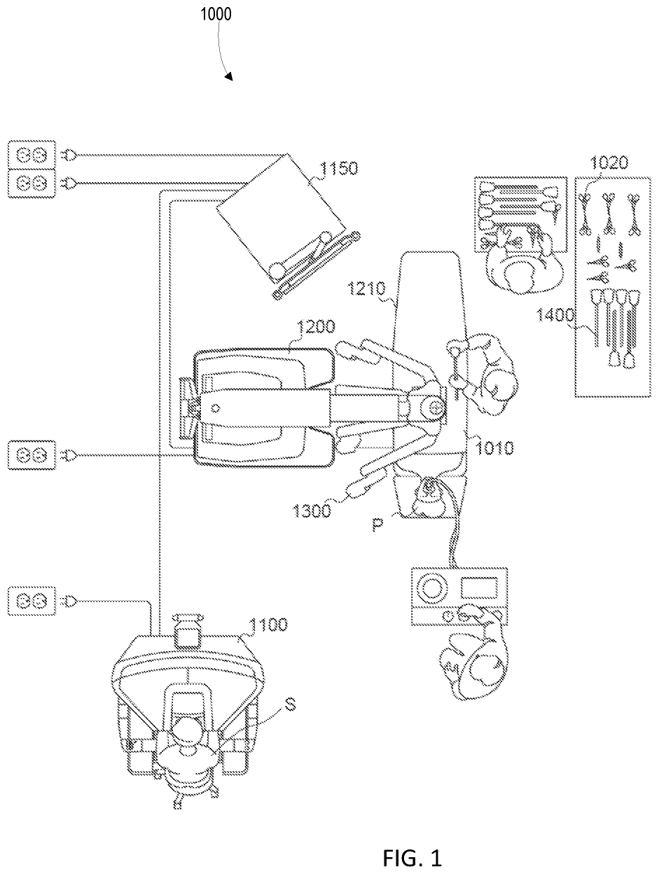

FIG. 1 is a plan view illustration of a computer-assisted teleoperation system. Shown is a medical device, which is a Minimally Invasive Robotic Surgical (MIRS) system 1000 (also referred to herein as a minimally invasive teleoperated surgery system—a telesurgical system), used for performing a minimally invasive therapeutic or diagnostic surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot), and an optional auxiliary equipment unit 1150. The manipulator unit 1200 can include an arm assembly 1300 and a tool assembly removably coupled to the arm assembly. The manipulator unit 1200 can manipulate at least one removably coupled instruments 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

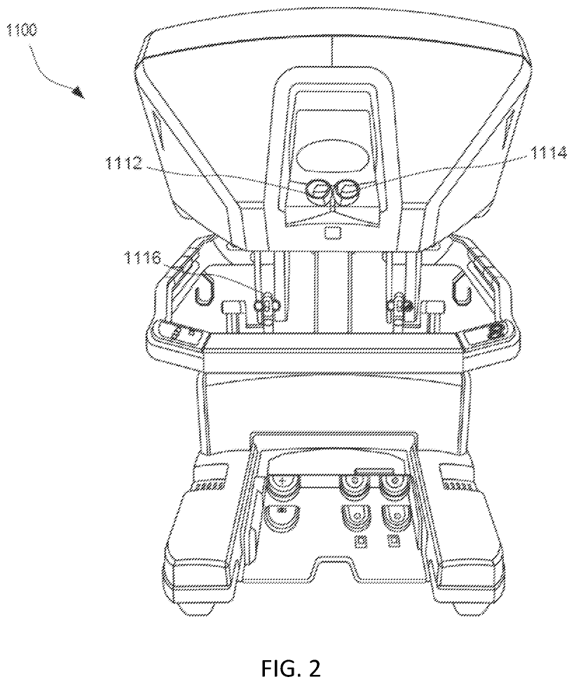

FIG. 2 is a perspective view of the control unit 1100. The user control unit 1100 includes a left eye display 1112 and a right eye display 1114 for presenting the surgeon S with a coordinated stereo view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain and/or tactile feedback sensors (not shown) may be employed to transmit position, force, and tactile sensations from the instruments 1400 back to the surgeon's hands through the input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other remote location from the patient allowing for remote surgical procedures.

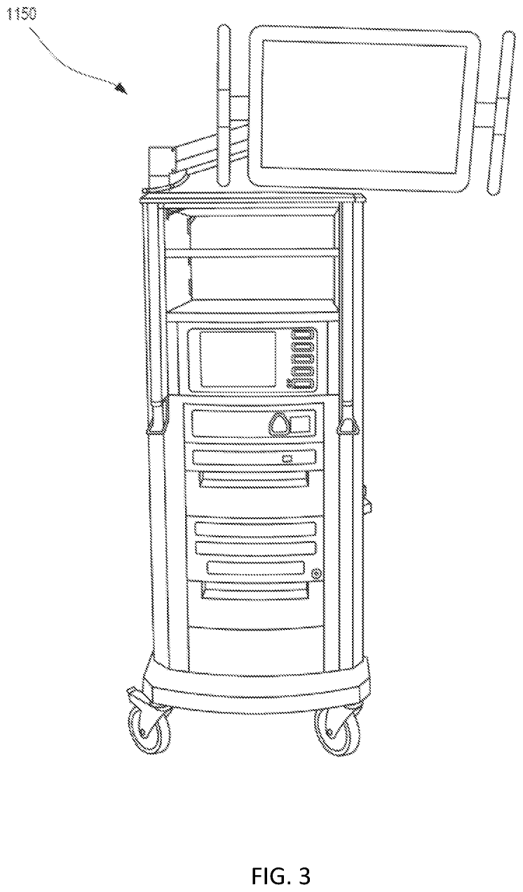

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

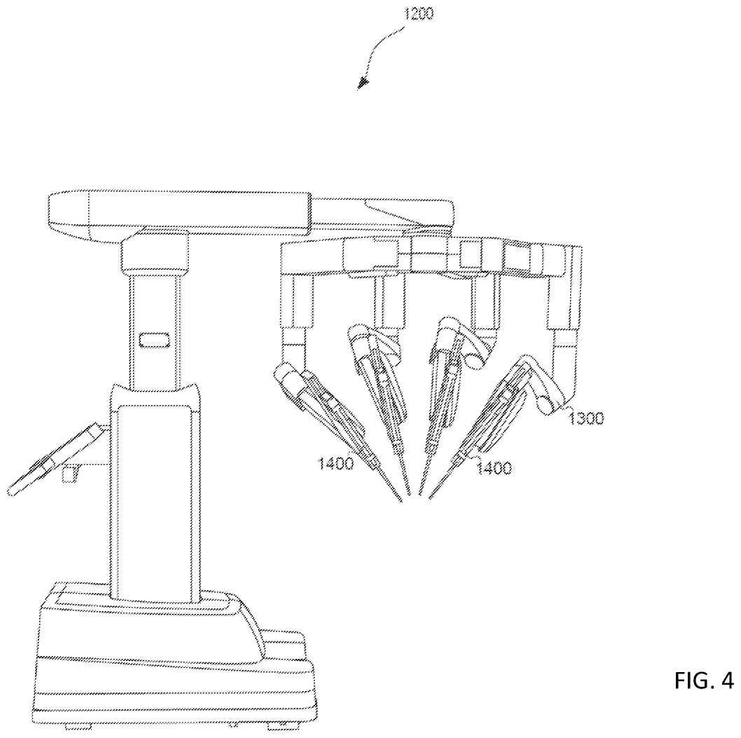

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having a number of joints. Moreover, the instruments 1400 and the imaging device are positioned and manipulated through incisions or natural orifices in the patient P in a manner such that a software and/or kinematic remote center of motion is maintained at the incision or orifice. In this manner, the incision size can be minimized.

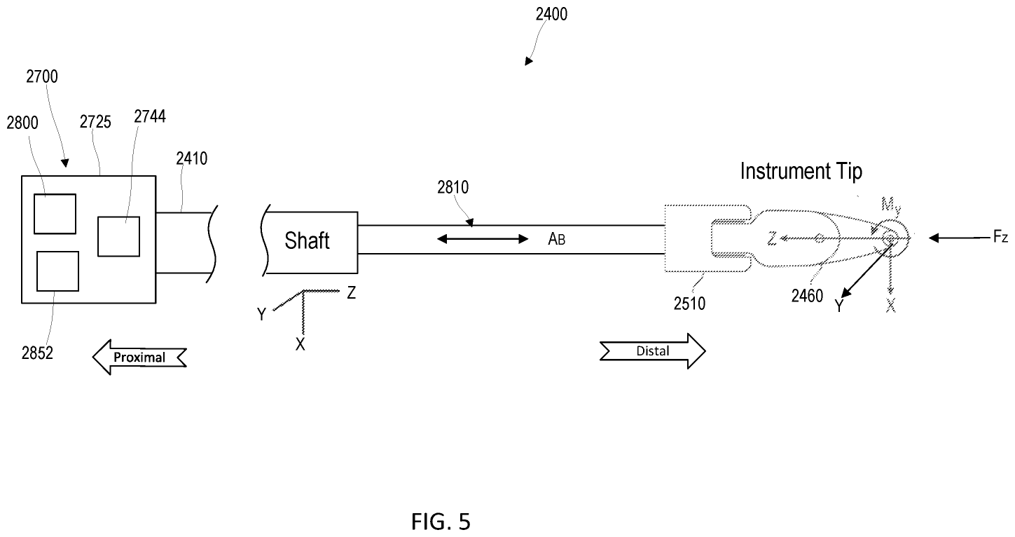

FIG. 5 is a schematic illustration of a medical device 2400, according to an embodiment. In some embodiments, the medical device 2400 or any of the components therein are optionally parts of a surgical system that performs surgical procedures, and which can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes a proximal mechanical structure 2700, a force sensor unit 2800 coupled to or included within the mechanical structure 2700, a shaft translation carriage 2852 coupled to or included within the mechanical structure 2700, a roll drive group 2744 coupled to or included within the mechanical structure 2700, a shaft 2410 coupled to the mechanical structure 2700, a beam 2810 coupled to the shaft 2410, and an end effector 2460 coupled at a distal end portion of the beam 2810. The end effector 2460 can include, for example, articulatable jaws or another suitable surgical tool that is coupled to a link 2510. In some embodiments, the link 2510 can be included within a wrist assembly having multiple articulating links. The shaft 2410 includes a distal end portion that is coupled to a proximal end portion of the beam 2810. In some embodiments, the distal end portion of the shaft 2410 is coupled to the proximal end portion 2822 of the beam 2810 via another coupling component (such as an anchor or coupler, not shown). The shaft 2410 is also movably coupled at a proximal end portion to the mechanical structure 2700. The mechanical structure 2700 can include components configured to move one or more components of the surgical instrument, such as, for example, the end effector 2460. The mechanical structure 2700 can be similar to the mechanical structure 5700 described in more detail below with reference to medical device 5400.

Generally, during a medical procedure, the end effector 2460 contacts anatomical tissue, which may result in X, Y, or Z direction forces being imparted on the end effector 2460 (see end effector reference frame in FIG. 5) and that may result in moment forces such as a moment My about a Y-direction axis as shown in FIG. 5. In some embodiments, one or more strain sensors (not shown), which can be electrical or optical strain gauges, can be coupled to the beam 2810 to measure strain in the beam 2810. The measured beam strain can be used to determine forces imparted on the end effector 2460 in the X- and Y-axis directions. These X- and Y-axis forces are transverse (e.g., perpendicular) to the Z axis (which is parallel or collinear with a center axis of the beam A_{B}). In some embodiments, the medical device 2400 is devoid of the beam 2810 and the sensors used to determine forces along the X and Y axes. In such embodiments, the link 2510 and/or the end effector 2460 can be directly coupled to the shaft 2410.

The X- and Y-axis forces transverse to the shaft's Z-axis (see shaft reference frame in FIG. 5) are sensed within the reference frame of the shaft. For example, the Z-axis forces are sensed proximally (i.e., via one or more sensors mounted at the proximal end of the instrument 2400) and the X- and Y-axis forces are sensed distally (i.e., via one or more sensors mounted at the proximal end of the instrument 2400), but the shaft reference frame does not change. Thus, an axial force imparted on the end effector 2460 will be the same as an axial force on the shaft 2410 when the end effector 2460 is axially aligned with the shaft 2410. Otherwise, an axial force imparted on the end effector 2460 will cause X- and Y-axis forces on the shaft 2410. In some cases, when the X-, Y-, and Z-axis forces on the shaft 2410 are known, force information on the end effector 2460 can be inferred because the position and orientation of the end effector 2460 with reference to the shaft 2410 are known. In some cases, forces sensed at the user input control is the Z-axis force along the shaft 2410 and the X- and Y-axis forces transverse to the shaft 2410.

The force sensor unit 2800 (and any of the force sensor units described herein) can be used to measure the axial force(s) (i.e., in the direction of the Z-axis parallel to the beam center axis A_{B}) imparted on the shaft 2410. For example, an axial force F_{Z} imparted to the end effector 2460 in a direction of the Z-axis can cause axial displacement of the shaft 2410 in a direction along a center axis of the shaft (substantially parallel to the beam center axis A_{B}). The axial force F_{Z} may be in the proximal direction (e.g., a reactive force resulting from pushing against tissue with the end effector) or it may be in the distal direction (e.g., a reactive force resulting from pulling tissue grasped with the end effector). As described herein, the shaft 2410 can be coupled to the mechanical structure 2700 via a biasing mechanism (e.g., a linkage or a spring-loaded coupling, not shown) such that the amount of travel of the shaft 2410 relative to the mechanical structure 2700 can be correlated to the magnitude of the axial force F_{Z} imparted to the end effector 2460. In this manner, measuring the distance through which the shaft 2410 moves relative to the mechanical structure 2700 can be used to determine the axial force F_{Z}.

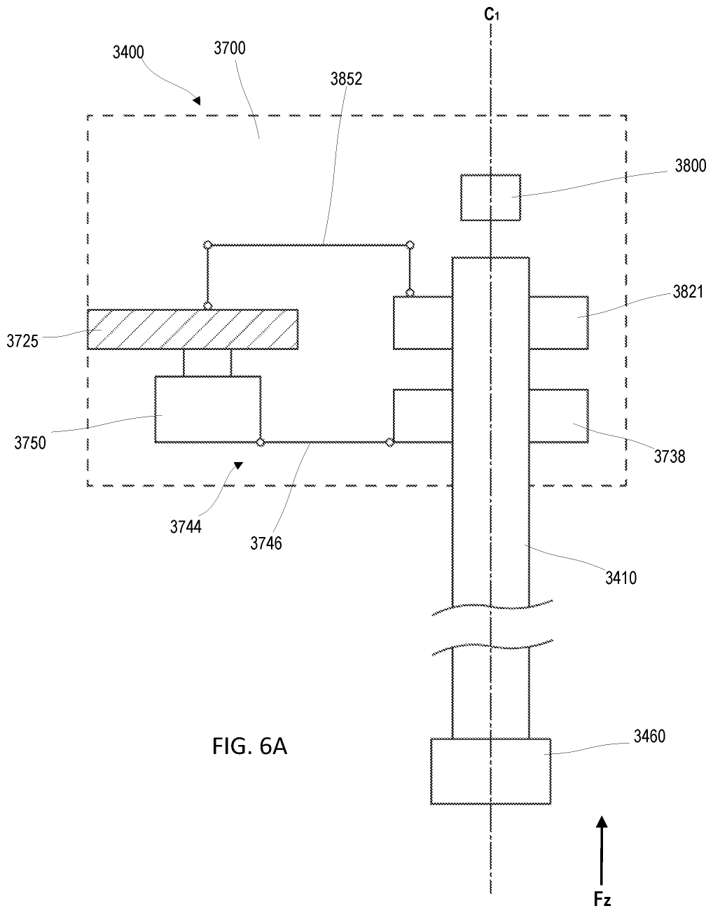

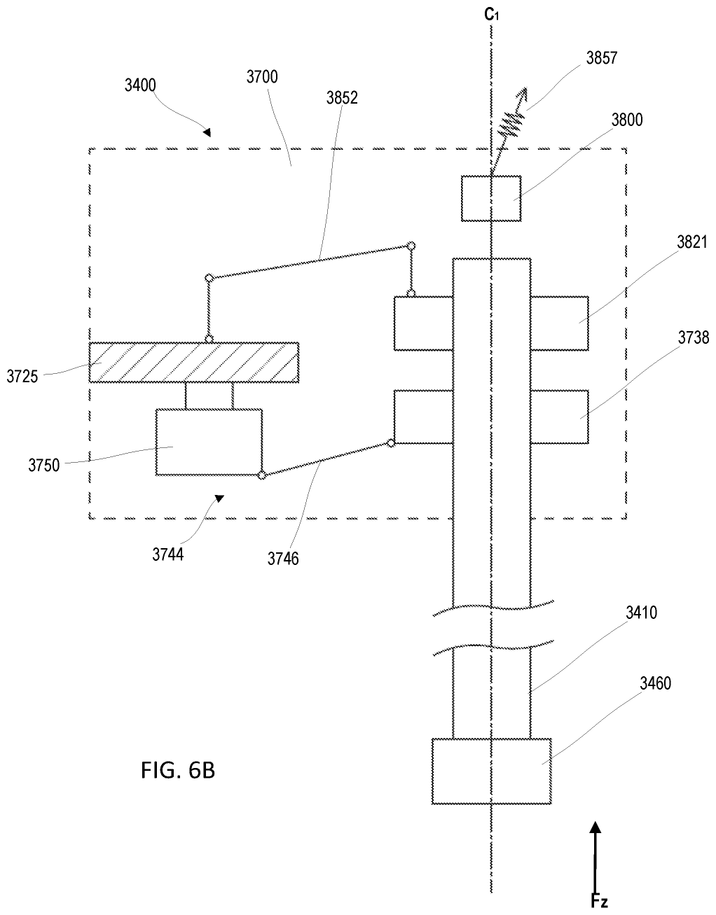

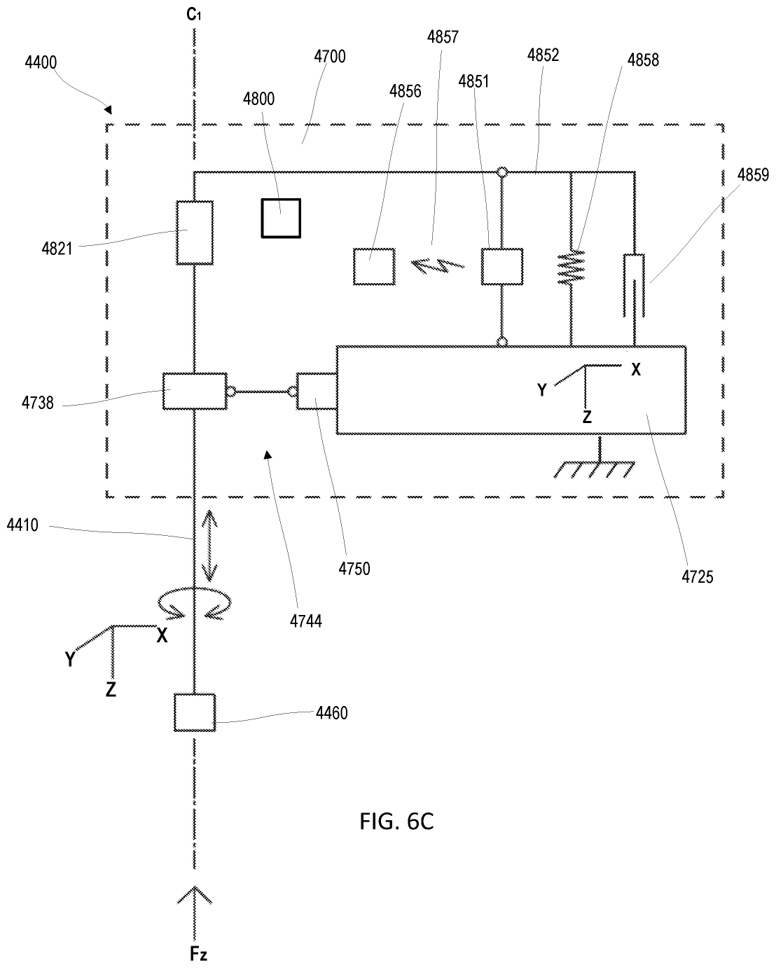

The shaft translation carriage 2852 can be any suitable mechanism that movably couples the shaft 2410 relative to the mechanical structure 2700 in a manner such that the amount of shaft movement can be correlated to the applied axial force F_{Z}. In some embodiments, the system is spring-biased such that the amount of axial force required to move the shaft translation carriage 2852 (and therefore the shaft 2410) through a given distance is correlated to the spring force. Similarly stated, in some embodiments, a resiliency is provided that can maintain the shaft 2410 at a defined location along the shaft axis. In other words, there is a lowest energy location along the shaft axis (or along the support structure reference frame's Z-axis (see, e.g., FIG. 6C)) to which the shaft tends, and the shaft translation in either direction away from this lowest energy location causes the resiliency to urge the shaft 2410 back towards the lowest energy location. As a result, the shaft 2410 floats at a defined location along the support structure reference frame's Z-axis. Thus, the resiliency (e.g., spring) is configured to be displaced in proportion to a force imparted to the shaft 2410 in a direction along the shaft axis and the resiliency counteracts the force applied at a distal end of the shaft 2410. The resiliency can be produced via any suitable arrangement. For example, in some embodiments the resiliency can include one or more springs coupled between a support structure 2725 of the mechanical structure 2700 and the shaft translation carriage 2852 or be provided as part of the shaft translation carriage 2852. The support structure 2725 can include a base and a top plate (each not shown in FIG. 5). In other embodiments, various support structures optionally may be used, such as a chassis, a frame, a bed, a unitized surrounding outer body of the mechanical structure, and the like. For example, in some embodiments, the resiliency can include a single spring that provides +Z forces and −Z forces, or the resiliency can include two or more springs that each provide +Z or −Z forces. In some embodiments, the resiliency is inherent in the shaft translation carriage 2852. For example, the shaft translation carriage 2852 can include components that provide a living hinge to allow for bending rotation of the shaft translation carriage 2852. Such an embodiment is shown and described with reference to medical device 7400 described below.

In this manner, the amount of travel of the shaft 2410 along the shaft axis for a given amount of axial force F_{Z} depends in part on the stiffness of the spring (or other structures) included within medical device 2400 that provide the resiliency as described above. Thus, the shaft translation carriage is calibrated to provide the desired range of motion of the shaft 2410 over the expected range of axial force. This arrangement can be used to translate (or correlate) the applied axial forces into a displacement signal.

In some embodiments, the shaft translation carriage 2852 is configured to constrain the movement of the shaft relative to the mechanical structure 2700 to translation along the shaft axis. Similarly stated, in some embodiments, the shaft translation carriage 2852 is configured to prevent tilting or “off-axis” movement of the shaft. This can be accomplished using any suitable structure. For example, in some embodiments, the shaft translation carriage 2852 includes two links and two translation flexures (not shown in FIG. 5) coupled together within the mechanical structure 2700 or coupled thereto. The translation flexures can provide the resiliency within the shaft translation carriage 2852. For example, in some embodiments, the shaft translation carriage 2852 can include a first link (which functions as a shaft roll carrier described herein) coupled to the shaft 2410, a second link that is fixed to the mechanical structure 2700, and two translation flexures coupled to the first link and the second link. The first link (not shown in FIG. 5) is configured to constrain shaft translation relative to the mechanical structure 2700 to translation along the shaft axis. The first link is also structured to constrain shaft roll relative to the mechanical structure to roll about the shaft axis. The two translation flexures provide a resiliency associated with the first link as described above and as described in more detail below with reference to specific embodiments.

In some embodiments, a medical device can include a shaft translation carriage 2852 that includes four links (not shown in FIG. 5) coupled together within or to the mechanical structure 2700. For example, a first link coupled to the shaft 2410, and a second link that includes or is coupled to a spring, a third link and a fourth link each pivotally coupled to the first link and the second link, as described in more detail below with reference to the linkage 5850. The four bar linkage configuration allows the shaft translation carriage 2852 to constrain the movement of the shaft relative to the mechanical structure 2700 to translation along the shaft axis. Persons of skill in the art will understand that for small translations along the shaft's Z-axis, concurrent transverse displacement of the shaft will be very small and may be ignored.

The components of the shaft translation carriage 2852 maintain connector tension (e.g., connectors used to move the end effector and wrist assembly, described in more detail below) within the medical device, and provide for linear movement of the shaft 2410 when forces are applied axially at the distal end of the medical device 2400. The shaft translation carriage 2852 also constrains the movement in the Z-axis and isolate forces in the Z-axis. As described below, the force sensor unit 2800 measures the Z-axis movement of the shaft, which is converted from a position measurement to a force measurement. The amount of travel of the shaft 2410 for a given amount of axial force F_{Z} depends in part on the stiffness of the resiliency (e.g., spring) included within the linkage.

The roll drive group 2744 is configured to rotate the shaft 2410 about a shaft axis to produce the roll DOF. In some embodiments, the roll drive group 2744 includes a shaft roll driver, a shaft roll drive receiver and a shaft roll drive coupling (each not shown in FIG. 5). The shaft roll driver is coupled to the mechanical structure 2700 and is operatively coupled to the shaft roll drive receiver with the shaft roll drive coupling. The shaft roll drive coupling can be, for example, a cable. The shaft roll driver is a motor-driven member that produces rotation of the shaft roll drive receiver, and in turn rotation of the shaft 2410. The shaft roll drive receiver is coupled to the shaft 2410 such that the shaft roll drive receiver also translates along the shaft axis (e.g., Z-axis) relative to the shaft roll driver as the shaft translates along the shaft axis. The first link of the shaft translation carriage 2852 also translates along the shaft axis with the shaft 2410 and is structured to constrain translation of the shaft 2410 relative to the support structure 2725 of the mechanical structure 2700 to translation along the shaft axis. Said another way, the shaft 2410 is coupled to the first link in a manner that restricts movement of the shaft 2410 relative to the first link along the Z-axis. The shaft roll driver is configured to cause the shaft roll drive receiver to rotate, which in turn causes the shaft 2410 (coupled thereto) to rotate relative to a base of the mechanical structure 2700, but the first link of the shaft translation carriage 2852 does not rotate with the shaft roll drive receiver and the shaft 2410. In other words, the first link of the shaft translation carriage 2852 translates along the shaft axis with the shaft 2410 but does not rotate with the shaft 2410. The shaft roll drive receiver can be actuated by the shaft roll drive coupling (e.g., a cable, band, cord or other suitable connector (not shown)) coupled to the shaft roll driver and wound about a portion of the shaft roll drive receiver as described in more detail below with reference to medical device 7400. This arrangement allows the shaft 2410 to move about the Z-axis relative to the mechanical structure 2700 (which allows measurement of the axial force) while also allowing the shaft 2410 to be rotated about the Z-axis.

The shaft translation carriage 2832 can include any suitable components to isolate the axial movement of the shaft 2410 (i.e., to constrain the shaft such that the measured movement is caused only by the axial force F_{Z} and not the transverse forces along the X and Y axes), and limit frictional force opposing movement of the shaft 2410 (which can cause errors in determining the axial force F_{Z}). The force sensor unit 2800 can include any suitable type of shaft translation sensors, such as, for example, various types of strain gauges, including hut not limited to conventional foil type electrical resistance gauges, semiconductor gauges, optic fiber type gauges using Bragg grating or Fably-Perot technology, an inductive coil force sensor, an electromagnetic sensor, or an optical sensor (e.g., time-of-flight (TOF)) or others, such as strain sensing surface acoustic wave (SAW) devices. Optic fiber Bragg grating (FBG) sensors may be advantageous in that two sensing elements may be located along one fiber at a known separation; thereby only requiring the provision of four fibers along the instrument shaft for eight gauges In some embodiments, the force sensor unit 2800 is incorporated within or coupled to the mechanical structure 2700. Foil type electrical resistance gauges may be advantageous because of relatively low cost and robustness to temperature changes, such as during surgery. Examples of foil type electrical resistance gauges are found in international Application No. PCT/US20201060636 (filed Nov. 15, 2020)(disclosing “Spread Bridge XY Sensor”), which is incorporated by reference in its entirety.

As described herein, the force sensor unit 2800 measures the displacement of the shaft along the Z-axis, which is then converted to the force measurement. In some embodiments, the force sensor unit 2800 can include a shaft translation sensor that includes a force flexure and an optical fiber sensor coupled thereto, and a microprocessor (each not shown in FIG. 5). The shaft translation sensor can be, for example, a Fiber Bragg Grating (FBG) optical fiber sensor or other types of force sensors as described herein. The shaft translation sensor is coupled to the force sensor flexure and is operatively coupled to a shaft translation information receiver (not shown in FIG. 5) that can receive the shaft translation information and can route that information for further processing to produce a haptic sensation force. The shaft translation information receiver can be incorporated into the medical device 2400 or be coupled thereto and can communicate with the shaft translation sensor. The force sensor flexure is coupled to the shaft 2410 via the first link of the shaft translation carriage such that as the shaft translates along the Z-axis, the force sensor flexure bends an amount that correlates to the amount of force imparted on the shaft 2410 as described in more detail herein.

More specifically, during use of the medical device 2400, as force is imparted on the shaft 2410 in a Z-direction, the shaft 2410 will travel along the Z-axis, which in turn causes a portion of the shaft translation carriage 2852 to translate along the Z-axis. The force sensor flexure and optical fiber sensor are coupled to the portion of the shaft translation carriage 2852 so that when the shaft 2410 moves axially due to forces imparted on a distal end of the medical device 2400, the force sensor flexure will deflect or bend an amount corresponding to a distance the shaft 2410 has traveled along the Z-axis. The optical fiber sensor is coupled to the force sensor flexure such that the amount of bend on the force sensor flexure is sensed by the optical fiber sensor, which can be translated to a Z-axis force measurement. A microprocessor receives the signals from the optical fiber sensor that is associated with a linear displacement of the shaft along the center axis of the shaft (e.g., along the Z-axis). The microprocessor is configured to execute instructions to determine a measure of a force on the shaft along the center axis of the shaft.

In alternative embodiments, the force sensor unit 2800 can include an inductive coil shaft translation sensor and a microprocessor (each not shown in FIG. 5). In such an embodiment, the inductive coil shaft translation sensor includes a coil assembly that can include two inductive coils each wound around a cylinder formed from a nonconductive material, such as, for example, PEEK. The two coils can be positioned side-by side to each other and coupled to, or within, the mechanical structure 2700. Within each of the coils is a rod that is movable within an interior of the coil and is coupled to the shaft 2410 of the medical device 2400. The rods can, for example, include a core with a magnet coupled to the core that moves with the rod within the respective coil. The core can be, for example, a glass core, a stainless steel core or a core formed with another suitable material. The magnet and any of the magnets described herein, can be, for example, a ferrite bead, an EMI suppression bead, a Nickel-zinc bead, or any other suitable material. Thus, it should be understood that the term “magnet” as used herein can refer to any component or material coupled to the core that can be used to provide a signal indicative of the position of the core within the coil as the rod and core move within the respective coil. The rods are operably coupled to the shaft 2410 such that when the shaft 2410 moves axially due to forces imparted on a distal end of the medical device 2400, the rods move with the shaft 2410 and within the coils. As the rods move within the inductive coils, the inductance at each of the coils changes, which can be used to measure changes in position of the instrument shaft. As described above, the change in position of the shaft 2410 can be translated to a Z-axis force measurement.

During use of the medical device 2400 having an inductive coil sensor, as force is imparted on the shaft 2410 in a Z-direction, the shaft 2410 will travel along the Z-axis, which in turn causes the rods to move along the Z-axis. As the rods move within the respective coils, each of the coils generate a signal associated with a position of the magnets of the rods within the respective coil. The microprocessor receives the signals from the coils. For example, in some embodiments, each of the coils generate a signal associated with a linear displacement of the shaft along the center axis of the shaft (e.g., along the Z-axis). In some embodiments, the signals from the coils can include a first signal from the first coil having a first frequency, and a second signal from the second coil having a second frequency. The microprocessor is configured to execute instructions to determine from the first frequency and the second frequency a measure of a force on the shaft along the center axis of the shaft.

FIGS. 6A and 6B are schematic illustrations of another embodiment of a medical device having a force sensor unit and a roll drive group to enable the shaft to rotate about a shaft axis. In some embodiments, the medical device 3400 or any of the components therein are optionally parts of a surgical system that performs surgical procedures, and which can include a manipulator unit, a series of kinematic linkages, a series of cannulas, or the like. The medical device 3400 (and any of the instruments described herein) can be used in any suitable surgical system, such as the MIRS system 1000 shown and described above. The medical device 3400 includes a mechanical structure 3700, a force sensor unit 3800, a shaft 3410 coupled to the mechanical structure 3700 and to the force sensor unit 3800, a shaft translation carriage 3852, a roll drive group 3744, and an end effector 3460 coupled at a distal end portion of the medical device 3400. As described above, the end effector 3460 can include, for example, articulatable jaws or another suitable surgical tool and can be coupled to a link (not shown). In some embodiments, the link can be included within a wrist assembly having multiple articulating links. The mechanical structure 3700 includes an instrument support structure 3725 that includes a base and a top plate (not shown). In other embodiments, various support structures optionally may be used, such as a chassis, a frame, a bed, a unitized surrounding outer body of the mechanical structure, and the like.

The shaft 3410 defines a shaft axis C₁ and includes a distal end portion that is coupled to the end effector 3460. In some embodiments, the distal end portion of the shaft 3410 is coupled to the end effector by a beam that can be used to measure transverse force applied thereto (e.g., similar to the beam 2810). In other embodiments, the shaft 3410 can be directly coupled to a link, wrist joint (not shown), or the end effector 3460. The shaft 3410 is movably coupled at a proximal end portion to the mechanical structure 3700. Thus, axial forces applied to the end effector 3460 will produce movement of the shaft 3410 relative to the mechanical structure 3700 (including the support structure 3725), which can be measured using the force sensor unit 3800, as described herein. The mechanical structure 3700 can include components configured to move one or more components of the surgical instrument, such as, for example, the end effector 3460. The mechanical structure 3700 can be similar to the mechanical structures 6700 and 5700 described in more detail below with reference to medical devices 7400 and 5400.

The shaft translation carriage 3852 can be any suitable mechanism that movably couples the shaft 3410 relative to the support structure 3725 of the mechanical structure 3700 in a manner such that the amount of shaft movement can be correlated to the applied axial force F_{Z}. As shown, the shaft translation carriage 3852 includes a first link 3821 (which functions as a shaft roll carrier) and one or more translation flexures (not shown in FIGS. 6A and 6B) coupled together within the mechanical structure 3700 or coupled thereto. The shaft translation carriage 3852 can also include a second link (not shown) that is fixed to the mechanical structure 3700 (which functions as a ground for the shaft translation carriage 3852). The first link 3821 is configured to constrain shaft translation relative to the support structure of the mechanical structure 3700 to translation along the shaft axis. Similarly stated, the first link 3821 is configured to prevent tilting or “off-axis” movement of the shaft 3410. The first link 3821 is also structured to constrain shaft roll relative to the support structure 3725 of the mechanical structure 3700 to roll about the shaft axis C₁.

The one or more translation flexures provide a resiliency associated with the first link 3821 that urges the shaft to a defined lowest energy location along the shaft axis C₁. Thus, the translation flexures (not shown in FIGS. 6A and 6B) can maintain the shaft 3410 at a defined location along the shaft axis C₁. In other words, there is a lowest energy location along the shaft axis C₁ (or Z-axis direction relative to the support structure 3725) to which the shaft tends, and the shaft translation in a direction away from this lowest energy location causes the resiliency to urge the shaft 3410 back towards the lowest energy location. As a result, the shaft 3410 floats at a defined location along the Z-axis. The resiliency (e.g., the translation flexures or spring) is configured to be displaced in proportion to a force imparted to the shaft 3410 in a direction along the shaft axis C₁ and the resiliency counteracts the force applied at a distal end of the shaft 3410. Although described as being produced by one or more translation flexures, the resiliency can be produced via any suitable arrangement. For example, in some embodiments the resiliency can include one or more springs coupled between the base 3770 and the shaft translation carriage 3852 or the provided as part of the shaft translation carriage 3852. For example, in some embodiments, the resiliency can include a single spring that provides +Z forces and −Z forces, or two springs that each provide +Z or −Z forces. In some embodiments, the resiliency is inherent in the shaft translation carriage 3852. For example, the shaft translation carriage 3852 can include components that provide a living hinge to allow for bending rotation of the shaft translation carriage 3852. Such an embodiment is shown and described with reference to medical device 7400 described below.

The amount of travel of the shaft 3410 for a given amount of axial force F_{Z} depends in part on the stiffness of the resiliency (e.g., spring, translation flexure, or the like) included within medical device 3400 that provides the biasing of the shaft 3410 as described above. Thus, the force sensor unit 3800 is calibrated to provide the desired range of motion of the shaft 3410 over the expected range of axial force. The resiliency can be used to translate the applied axial forces into a displacement signal.

The roll drive group 3744 is configured to rotate the shaft 3410 about the shaft axis C₁ and includes a shaft roll driver 3750, a shaft roll drive receiver 3738 and a shaft roll drive coupling 3746. The shaft roll driver 3750 is coupled to the support structure 3725 of the mechanical structure 3700 and is operatively coupled to the shaft roll drive receiver 3738 via the shaft roll drive coupling 3746. The shaft roll drive coupling 3746 can be, for example, a cable, a belt, or a gear. The shaft roll driver 3750 is a motor-driven member that produces rotation of the shaft roll drive receiver 3738, and in turn rotation of the shaft 3410. The shaft roll drive receiver 3738 is also coupled to the shaft 3410 such that the shaft roll drive receiver 3738 translates along the shaft axis C₁ (e.g., Z-axis) relative to the shaft roll driver 3750 as the shaft 3410 translates along the shaft axis C₁. The first link 3821 of the shaft translation carriage 3852 also translates along the shaft axis C₁ and is structured to constrain translation of the shaft 3410 relative to the support structure of the mechanical structure 3700 to translation along the shaft axis C₁. Said another way, the shaft 3410 is coupled to the first link 3821 in a manner that restricts movement of the shaft 3410 relative to the first link 3821 along the Z-axis. The shaft roll driver 3750 is configured to cause the shaft roll drive receiver 3738 to rotate, which in turn causes the shaft 3410 (coupled thereto) to rotate relative to the support structure 3725 of the mechanical structure 3700. The first link 3821 of the shaft translation carriage 3852, however, does not rotate with the shaft roll drive receiver 3738 and the shaft 3410. In other words, the first link 3821 of the shaft translation carriage 3852 translates along the shaft axis C₁ with the shaft 3410, but does not rotate with the shaft 3410. The shaft roll drive receiver 3738 can be actuated by the shaft roll drive coupling 3746 (e.g., a cable, band, cord, gear or other suitable connector) coupled to the shaft roll driver 3750. In some embodiments, the shaft roll drive coupling 3746 is a cable wound about a portion of the shaft roll drive receiver 3738 as described in more detail below with reference to medical device 7400. This arrangement allows the shaft 3410 to move about the Z-axis relative to the mechanical structure 3700 (which allows measurement of the axial force) while also allowing the shaft 3410 to be rotated about the Z-axis.

FIG. 6A illustrates the medical device 3400 prior to a Z-axis force F_{Z} being imparted on the shaft 3410 and FIG. 6B illustrates the medical device 3400 when a Z-axis force F_{Z} is imparted on the distal end portion of the medical device 3400. As shown, when the force F_{Z} is imparted on the shaft 3410, the shaft 3410 is moved in a direction along the shaft axis C₁, and the shaft translation carriage 3852, including the first link 3821 also moves in the same direction, and the resiliency in the shaft translation carriage 3852 allows the shaft translation carriage 3852 to move (or bend) an amount proportionate to the displacement of the shaft 3410. As shown in FIG. 6B, the shaft roll drive receiver 3738 is coupled to the shaft 3410 such that the shaft roll drive receiver 3738 moves with the shaft 3410 as the shaft 3410 translates along the shaft axis C₁.

The shaft translation carriage 3852 includes any suitable components to isolate the axial movement of the shaft 3410 (i.e., to constrain the shaft such that the measured movement is caused only by the axial force F_{Z} and not the transverse forces along the X and Y axes), and limit frictional force opposing movement of the shaft 3410 (which can cause errors in determining the axial force F_{Z}). The force sensor unit 3800 can include any suitable type of shaft translation sensors, such as, for example, various types of strain gauges, including but not limited to conventional foil type resistance gauges, semiconductor gauges, optic fiber type gauges using Bragg grating or Fabry-Perot technology, an inductive coil force sensor, an electromagnetic sensor, or an optical sensor (e.g., time-of-flight (TOF)) or others, such as strain sensing surface acoustic wave (SAW) devices. In some embodiments, the force sensor unit 3800 is incorporated within or coupled to the mechanical structure 3700. The shaft translation sensor of the force sensor unit 3800 measures the displacement of the shaft 3410 along the Z-axis, which is then converted to the force measurement. The shaft translation sensor can be coupled to a shaft translation information receiver (not shown in FIGS. 6A and 6B) that can receive the shaft translation information 3857 (FIG. 6B) and can route that information for further processing to produce a haptic sensation force. The shaft translation information receiver can be incorporated into the medical device 3400 or be coupled thereto and can communicate with the force sensor.

In some embodiments, the force sensor unit 3800 includes a shaft translation sensor coupled to a force sensor flexure (each not shown in FIGS. 6A and 6B) that is coupled to the shaft 3410 via the first link 3821 of the shaft translation carriage 3852 such that as the shaft 3410 translates along the Z-axis, the force sensor flexure bends an amount that correlates to the amount of force imparted on the shaft 3410 as described in more detail herein. The force sensor unit 3800 can also include a microprocessor (not shown in FIGS. 6A and 6B) that receives the signals from the shaft translation sensor that is associated with a linear displacement of the shaft 3410 along the shaft axis C₁ of the shaft (e.g., along the Z-axis). The microprocessor is configured to execute instructions to determine a measure of a force on the shaft along the shaft axis of the shaft. In alternative embodiments, the force sensor unit 3800 can include an induction coil sensor, a linkage (as described above), and a microprocessor (each not shown in FIGS. 6A and 6B).

FIG. 6C is a schematic illustration of another embodiment of a medical device 4400 having a force sensor unit and a roll drive group to enable a shaft to rotate about a shaft axis while also allowing translation of the shaft to measure the force imparted on an end effector. In some embodiments, the medical device 4400 or any of the components therein are optionally parts of a surgical system that performs surgical procedures, and which can include a manipulator unit, a series of kinematic linkages, a series of cannulas, or the like. The medical device 4400 (and any of the instruments described herein) can be used in any suitable surgical system, such as the MIRS system 1000 shown and described above. The medical device 4400 includes a mechanical structure 4700, a force sensor unit 4800, a shaft 4410 coupled to the mechanical structure 4700 and, at least operatively, to the force sensor unit 4800, a shaft translation carriage 4852, a roll drive group 4744, and an end effector 4460 coupled at a distal end portion of the medical device 4400. As described above, the end effector 4460 can include, for example, articulatable jaws or another suitable surgical tool and can be coupled to a link (not shown). In some embodiments, the link can be included within a wrist assembly having multiple articulating links. The mechanical structure 4700 includes an instrument support structure 4725 that can include, for example, a base and a top plate (each not shown in FIG. 6C). As shown, the support structure 4725 defines a support structure reference frame, shown as the X, Y, and Z-axes within the support structure 4725.

The shaft 4410 defines a shaft axis C₁ and includes a distal end portion that is coupled to the end effector 4460. In some embodiments, the distal end portion of the shaft 4410 is coupled to the end effector 4460 by a beam (not shown) that can be used to measure transverse force applied thereto (e.g., similar to the beam 2810). In other embodiments, the shaft 4410 can be directly coupled to a link, wrist joint (not shown), or the end effector 4460. The shaft 4410 is movably coupled at a proximal end portion to the mechanical structure 4700. Thus, axial forces applied to the end effector 4460 will produce movement of the shaft 4410 relative to the mechanical structure 4700 (and the support structure reference frame), which can be measured using the force sensor unit 4800, as described herein. The mechanical structure 4700 can include components configured to move one or more components of the surgical instrument, such as, for example, the end effector 4460. The mechanical structure 4700 can be similar to the mechanical structures 6700 and 5700 described in more detail below with reference to medical devices 7400 and 5400.

The shaft translation carriage 4852 can be any suitable mechanism that movably couples the shaft 4410 relative to the support structure 4725 of the mechanical structure 4700 in a manner such that the amount of shaft movement can be correlated to the applied axial force F_{Z}. The shaft translation carriage 4852 includes a resiliency 4858 and can provide a shaft translation degree of freedom (DOF) with reference to the support structure 4725 represented as 4859 in FIG. 6C. As described above, the resiliency 4858 can be provided in the form of one or more springs coupled between the support structure 4725 of the mechanical structure 4700 and the shaft translation carriage 4852, one or more flexures, or can be provided as part of the shaft translation carriage 4852 (or as a combination of any of these). The resiliency 4858 can maintain the shaft 4410 at a defined location along the shaft axis. In other words, there is a lowest energy location along the shaft axis C₁ (or along the support structure reference frame's Z-axis) to which the shaft tends, and the shaft translation in a direction away from this lowest energy location causes the resiliency 4858 to urge the shaft 4410 back towards the lowest energy location. As a result, the shaft 4410 floats at a defined location along the support structure reference frame's Z-axis. Thus, the resiliency 4858 (e.g., spring, flexure, or the like) is configured to be displaced in proportion to a force imparted to the shaft 4410 in a direction along the shaft axis C₁ and the resiliency 4858 counteracts the force F_{Z} applied at a distal end of the shaft 4410. Various spring arrangements can be used to provide the resiliency 4858. For example, a single spring that provides +Z forces and −Z forces, or two springs that each provide +Z or −Z forces. In some embodiments, the resiliency 4858 is inherent in the shaft translation carriage 4852. For example, the shaft translation carriage 4852 can include components that provide a living hinge to allow for bending rotation of the shaft translation carriage 4852. Such an embodiment is shown and described with reference to medical device 7400 described below.

The amount of travel of the shaft 4410 for a given amount of axial force F_{Z} depends in part on the stiffness of the resiliency 4858 (e.g., spring, flexure, or the like), as described above. Thus, the shaft translation carriage 4852 is calibrated to provide the desired range of motion of the shaft 4410 over the expected range of axial force F_{Z}. This arrangement can be used to translate (or correlate) the applied axial forces into a displacement signal.

In some embodiments, the shaft translation carriage 4852 includes a first link 4821 (which functions as a shaft roll carrier) The shaft translation carriage 4852 is configured to constrain shaft translation relative to the support structure of the mechanical structure 4700 to translation along the shaft axis C₁. More particularly, as shown by the shaft translation degree of freedom (DOF) 4859, the shaft translation carriage 4852 is configured to constrain the shaft 4410 to prevent tilting or “off-axis” movement of the shaft 4410 when it is moved due to the axial force F_{Z}. The shaft translation carriage 4852 is also structured to constrain shaft roll relative to the support structure 4725 of the mechanical structure 4700 to roll about the shaft axis C₁.

The roll drive group 4744 is configured to rotate the shaft 4410 about the shaft axis C₁ and includes a shaft roll driver 4750, a shaft roll drive receiver 4738 and a shaft roll drive coupling 4746. The shaft roll driver 4750 is coupled to the support structure 4725 of the mechanical structure 4700 and is operatively coupled to the shaft roll drive receiver 4738 via the shaft roll drive coupling 4746. The shaft roll drive coupling 4746 can be, for example, a cable a belt, a gear, or the like. The shaft roll driver 4750 is a motor-driven member that produces rotation of the shaft roll drive receiver 4738, and in turn rotation of the shaft 4410. The shaft roll drive receiver 4738 is also coupled to the shaft 4410 such that the shaft 4410 can translate along the shaft axis C₁ (e.g., Z-axis) relative to the shaft roll driver 4750 as the shaft 4410 translates along the shaft axis C₁. A portion of the shaft translation carriage 4852 also translates along the shaft axis C₁ and is structured to constrain translation of the shaft 4410 relative to the support structure of the mechanical structure 4700 to translation along the shaft axis C₁. Said another way, the shaft 4410 is coupled to the shaft translation carriage 4852 in a manner that restricts movement of the shaft 4410 relative to the shaft translation carriage 4852 along the Z-axis. The shaft roll driver 4750 is configured to cause the shaft roll drive receiver 4738 to rotate, which in turn causes the shaft 4410 (coupled thereto) to rotate relative to the support structure 4725 of the mechanical structure 4700. The shaft translation carriage 4852 and the first link 4821, however, do not rotate with the shaft roll drive receiver 4738 and the shaft 4410. In other words, the first link 4821 translates along the shaft axis C₁ with the shaft 4410, but does not rotate with the shaft 4410. The shaft roll drive receiver 4738 can be actuated by the shaft roll drive coupling 4746 (e.g., a cable, band, cord or other suitable connector coupled to the shaft roll driver 4750). In some embodiments, the shaft roll drive coupling 4746 can be a cable that is wound about a portion of the shaft roll drive receiver 4738 as described in more detail below with reference to medical device 7400. This arrangement allows the shaft 4410 to move about the Z-axis relative to the mechanical structure 4700 (which allows measurement of the axial force) while also allowing the shaft 4410 to be rotated about the Z-axis.

The shaft translation carriage 4852 includes any suitable components to produce the shaft translation degree of freedom (DOF) 4859. Similarly stated, the shaft translation carriage 4852 includes any suitable components to isolate the axial movement of the shaft 4410 (i.e., to constrain the shaft such that the measured movement is caused only by the axial force F_{Z} and not the transverse forces along the X and Y axes), and limit frictional force opposing movement of the shaft 4410 (which can cause errors in determining the axial force F_{Z}). The force sensor unit 4800 can include any suitable types of shaft translation sensor(s) 4851, such as, for example, various types of strain gauges, including but not limited to conventional foil type resistance gauges, semiconductor gauges, optic fiber type gauges using Bragg grating or Fabry-Perot technology, an inductive coil force sensor, an electromagnetic sensor, or an optical sensor (e.g., time-of-flight (TOF)) or others, such as strain sensing surface acoustic wave (SAW) devices. Optic fiber Bragg grating (FBG) sensors may be advantageous in that two sensing elements may be located along one fiber at a known separation, thereby only requiring the provision of four fibers along the instrument shaft for eight gauges. In some embodiments, the force sensor unit 4800 is incorporated within or coupled to the mechanical structure 4700.

The shaft translation sensor 4851 of the force sensor unit 4800 measures the displacement of the shaft 4410 along the Z-axis, which is then converted to the force measurement. In some embodiments, the shaft translation sensor 4851 can be, for example, a Fiber Bragg Grating (FBG) optical fiber sensor or other types of force sensors as described herein. The shaft translation sensor 4851 can be coupled to a shaft translation information receiver 4856 that can receive shaft translation information 4857 and can route that information for further processing to produce a haptic sensation force. The shaft translation information receiver 4856 can be incorporated into the medical device 4400 or be coupled thereto and can communicate with the shaft translation sensor 4851.

In some embodiments, the force sensor unit 4800 includes a shaft translation sensor 4851 coupled to a force sensor flexure (not shown in FIG. 6C) that is coupled to the shaft 4410 via the shaft translation carriage 4852 such that as the shaft 4410 translates along the Z-axis, the force sensor flexure bends an amount that correlates to the amount of force imparted on the shaft 4410. The force sensor unit 4800 can also include a microprocessor (not shown in FIG. 6C) that receives the signals from the shaft translation sensor 4851 that is associated with a linear displacement of the shaft 4410 along the shaft axis C₁ of the shaft (e.g., along the Z-axis). The microprocessor is configured to execute instructions to determine a measure of a force on the shaft along the shaft axis. In alternative embodiments, the force sensor unit 4800 can include an induction coil sensor (not shown in FIG. 6C), as described above.

The amount of travel of the shaft 4410 for a given amount of axial force F_{Z} depends in part on the stiffness of the resiliency 4858 included within the shaft translation carriage 4852. Thus, as described above for previous embodiments, the force sensor unit 4800 is calibrated to provide the desired range of motion of the shaft 4410 over the expected range of axial force. The spring can translate the applied axial forces into a displacement signal.

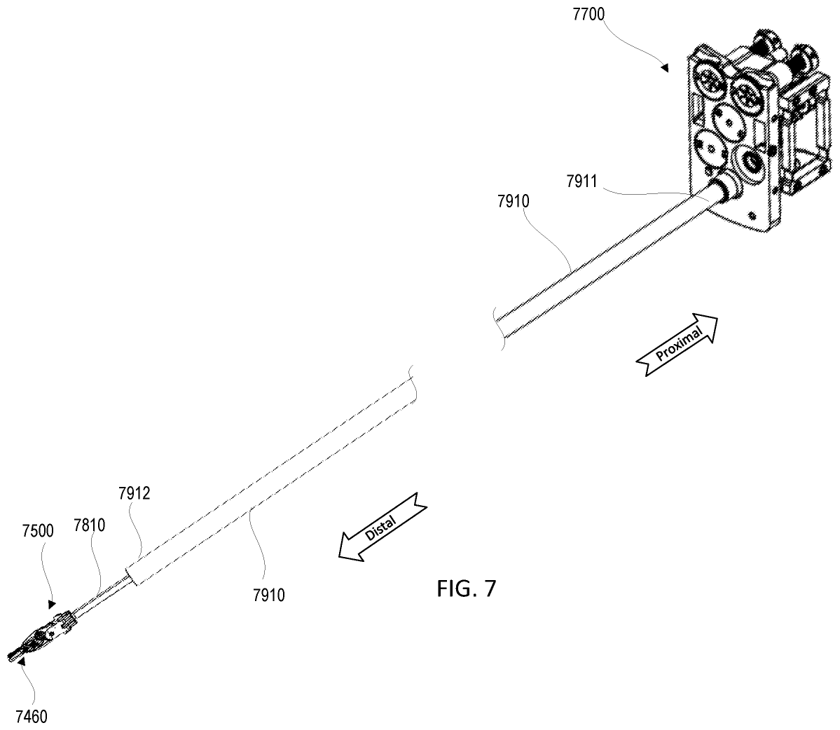

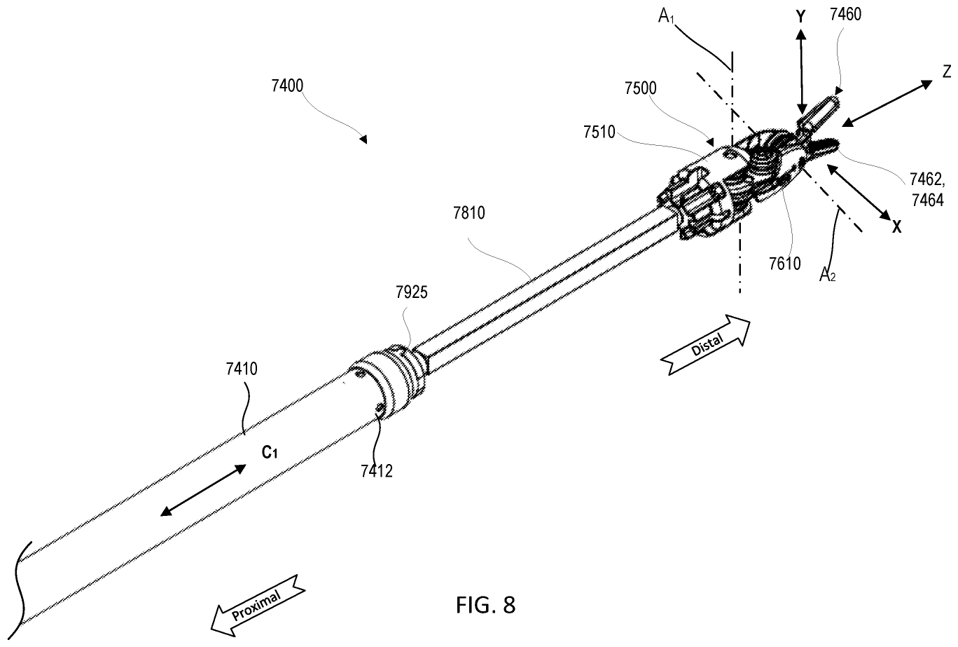

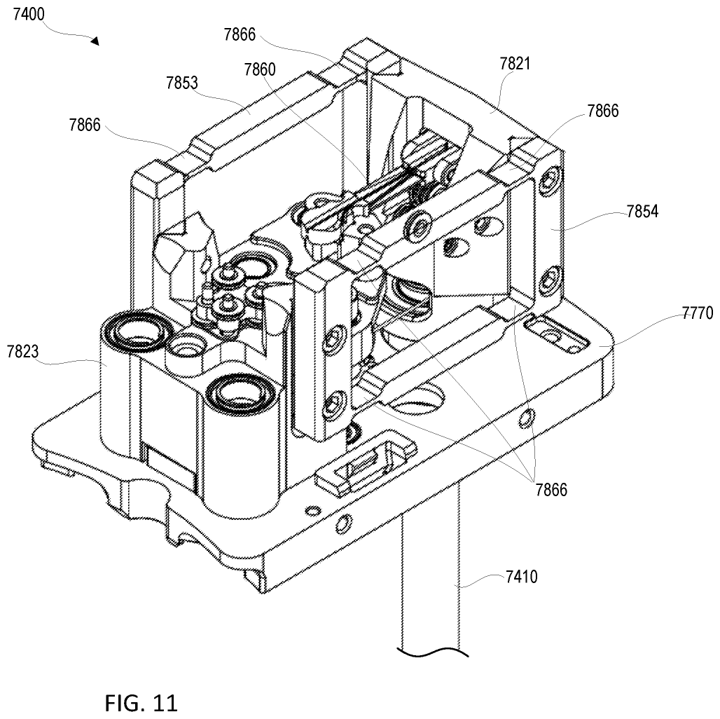

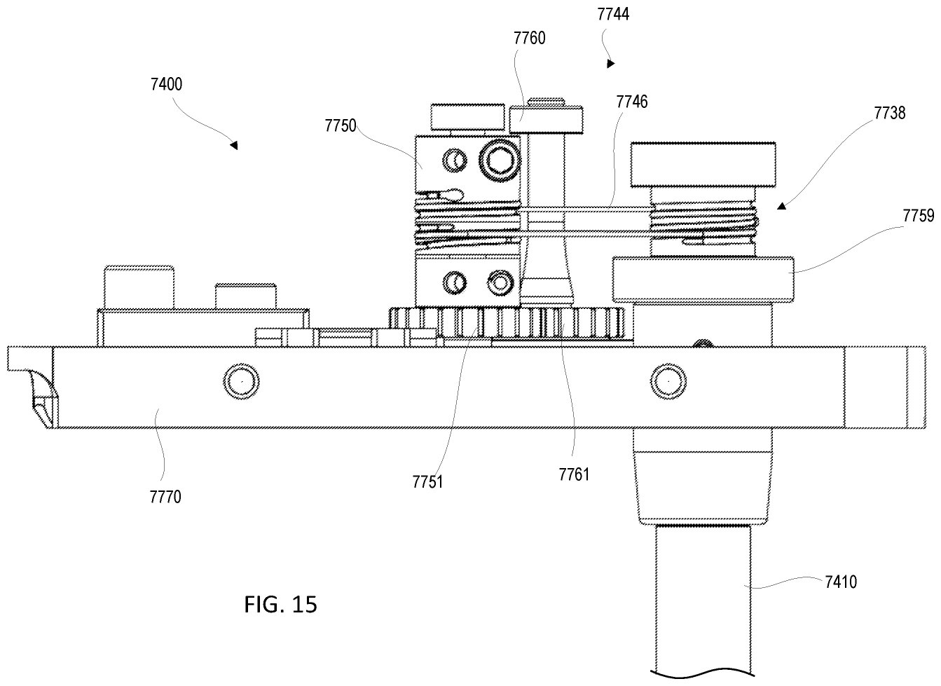

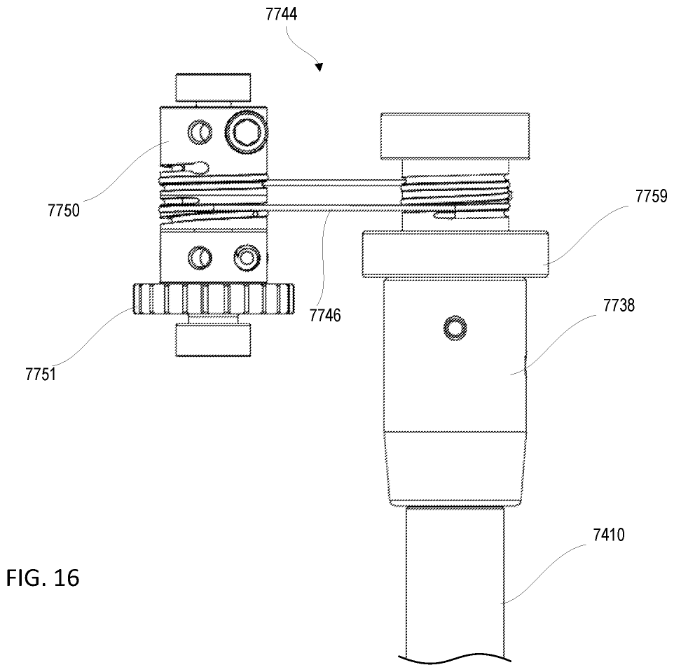

FIGS. 7-16 are various views of a medical device 7400 and its components, according to an embodiment. In some embodiments, the medical device 7400 or any of the components therein are optionally parts of a surgical system that performs surgical procedures, and which can include a manipulator unit, a series of kinematic linkages, a series of cannulas, or the like. The medical device 7400 (and any of the instruments described herein) can be used in any suitable surgical system, such as the MIRS system 1000 shown and described above. The medical device 7400 includes a mechanical structure 7700 at a proximal end portion of the medical device 7400, an outer shaft 7910, a shaft 7410 (which functions as an inner shaft in this embodiment), a force sensor unit 7800, a shaft translation carriage 7852, a shaft roll drive group 7744 (see FIGS. 15 and 16), and a distal end mechanism which includes a wrist assembly 7500, and an end effector 7460. Although not shown, the medical device 7400 can also include one or more connectors that couple the mechanical structure 7700 to the wrist assembly 7500 and end effector 7460, and function as tension members to actuate the end effector 7460. In some embodiments, the connectors can be a cable, a band or the like. The instrument 7400 is configured such that select movements of the connectors produces rotation of the wrist assembly 7500 (i.e., pitch rotation) about a first axis of rotation A₁ (see FIG. 8) (which functions as a pitch axis; the term pitch is arbitrary), yaw rotation of the end effector 7460 about a second axis of rotation A₂ (see FIG. 8) (which functions as the yaw axis; the term yaw is arbitrary), a cutting rotation of the tool members of the end effector 7460 about the second axis of rotation A₂, or any combination of these movements. Changing the pitch or yaw of the instrument 7400 can be performed by manipulating the connectors in a similar manner as described, for example, in U.S. Pat. No. 8,821,480 B2 (filed Jul. 16, 2008), entitled “Four-Cable Wrist with Solid Surface Cable Channels,” which is incorporated herein by reference in its entirety. Thus, the specific movement of each of the connectors to accomplish the desired motion is not described below.

FIGS. 8 and 10-12 are shown with the outer shaft 7910 removed to clearly show the inner shaft 7410. The shaft 7410 includes a proximal end 7411 that is coupled to the mechanical structure 7700, and a distal end 7412 (see FIG. 8) that is coupled to a beam 7810 via an anchor 7925. The beam 7810 can include or have coupled thereto one or more strain sensors (not shown) to measure forces imparted on the surgical instrument in the X and Y direction during a surgical procedure. Thus, the beam 7810 can be a part of a force sensor unit similar those shown and described in co-pending U.S. Provisional Patent Application No. 63/026,321 (filed May 18, 2020), entitled “Devices and Methods for Stress/Strain Isolation on a Force Sensor Unit,” the disclosure of which is incorporated herein by reference in its entirety. Although a beam 7810 with X-Y sensors is shown and described in this embodiment, in other embodiments, a beam 7810 and X-Y sensors may not be included. The proximal end of the shaft 7410 is coupled to the mechanical structure 7700 in a manner that allows movement of the shaft 7410 along a shaft axis C₁ of the shaft 7410 (shown in FIG. 8) relative to the mechanical structure 7700. More specifically, in this embodiment, the shaft 7410 extends through an opening (not shown) in a base 7770 of the mechanical structure 7700 and is coupled to a first link 7821 of shaft translation carriage 7852 that allows for the shaft 7410 to translate in the Z-axis direction and also rotate, as described in more detail below. Allowing the shaft 7410 to “float” in the Z direction facilitates measurement of forces along the Z axis, as described herein. The shaft 7410 also defines a lumen (not shown) and/or multiple passageways through which the connectors and other components (e.g., electrical wires, ground wires, or the like) can be routed from the mechanical structure 7700 to the wrist assembly 7500. The anchor 7925 can be received at least partially within the lumen of the shaft 7410 and can be fixedly coupled to the shaft 7410 via an adhesive bond, a weld, or any other permanent coupling mechanism (i.e., a coupling mechanism that is not intended to be removed during normal use).

The outer shaft 7910 can be any suitable elongated shaft that can be disposed over the shaft 7410 and includes a proximal end 7911 that can be coupled to the mechanical structure 7700 and a distal end 7912. The outer shaft 7910 defines a lumen between the proximal end 7911 and the distal end 7912. The shaft 7410 extends within the lumen of the outer shaft 7910 and can move relative to the outer shaft 7910. For example, the shaft 7410 can rotate relative to the outer shaft 7910 and/or can translate longitudinally in a direction parallel to the shaft axis C₁ of the shaft 7410 (i.e., the Z-direction).

Referring to FIG. 8, the wrist assembly 7500 includes a proximal first link 7510 and a distal second link 7610. The first link 7510 includes a distal portion that is coupled to a proximal portion of the second ink 7610 at a joint such that the second link 7610 can rotate relative to the first link 7510 about a first axis of rotation A₁ (which functions as the pitch axis, the term pitch is arbitrary). The proximal first link 7510 includes a proximal portion that is coupled to the beam 7810 as described in more detail herein.

A distal end of the distal second link 7610 is coupled to the end effector 7460 such that the end effector 7460 can rotate about a second axis of rotation A₂ (see FIG. 8) (which functions as the yaw axis). The end effector 7460 can include at least one tool member 7462 having a contact portion 7464 configured to engage or manipulate a target tissue during a surgical procedure. For example, in some embodiments, the contact portion 7464 can include an engagement surface that functions as a gripper, cutter, tissue manipulator, or the like. In other embodiments, the contact portion 7464 can be an energized tool member that is used for cauterization or electrosurgical procedures. The end effector 7460 is operatively coupled to the mechanical structure 7700 such that the tool member 7462 rotates relative to shaft 7410 about the first axis of rotation A₁. In this manner, the contact portion 7464 of the tool member 7462 can be actuated to engage or manipulate a target tissue during a surgical procedure. The tool member 7462 (or any of the tool members described herein) can be any suitable medical tool member. Moreover, although only one tool member 7462 is identified, as shown, the instrument 7400 can include two tool members that cooperatively perform gripping or shearing functions. In other embodiments, an end effector can include more than two tool members.

The mechanical structure 7700 includes components to produce movement of the connectors (not shown) to produce the desired movement (pitch, yaw, or grip) at the wrist assembly 7500. Specifically, the mechanical structure 7700 includes components and controls to move some of the connectors in a proximal direction (i.e., to pull in certain connectors) while simultaneously allowing the distal movement (i.e., releasing or “paying out”) of other of the connectors in equal lengths. In this manner, the mechanical structure 7700 can maintain the desired tension within the connectors, and in some embodiments, can ensure that the lengths of the connectors are conserved (i.e., moved in equal amounts) during the entire range of motion of the wrist assembly 7500. In other embodiments, however, conservation of the lengths of the connectors is not required.

In some embodiments, the mechanical structure 7700 can include one or more mechanisms that produce translation (linear motion) of a portion of the connectors. Such a mechanisms can include, for example, a gimbal, a lever, or any other suitable mechanism to directly pull (or release) an end portion of any of the connectors. For example, in some embodiments, the mechanical structure 7700 can include any of the mechanical structures (referred to as backend assemblies or actuators) or components described in U.S. Patent Application Pub. No. US 20157/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

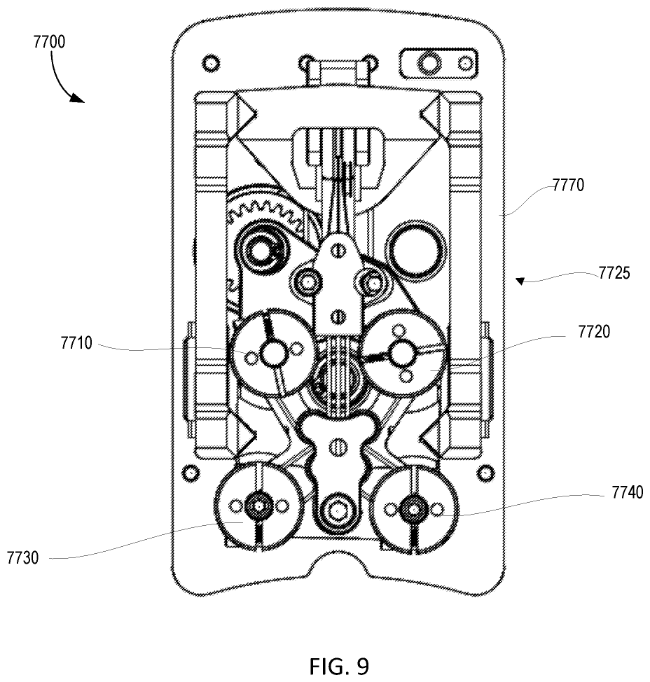

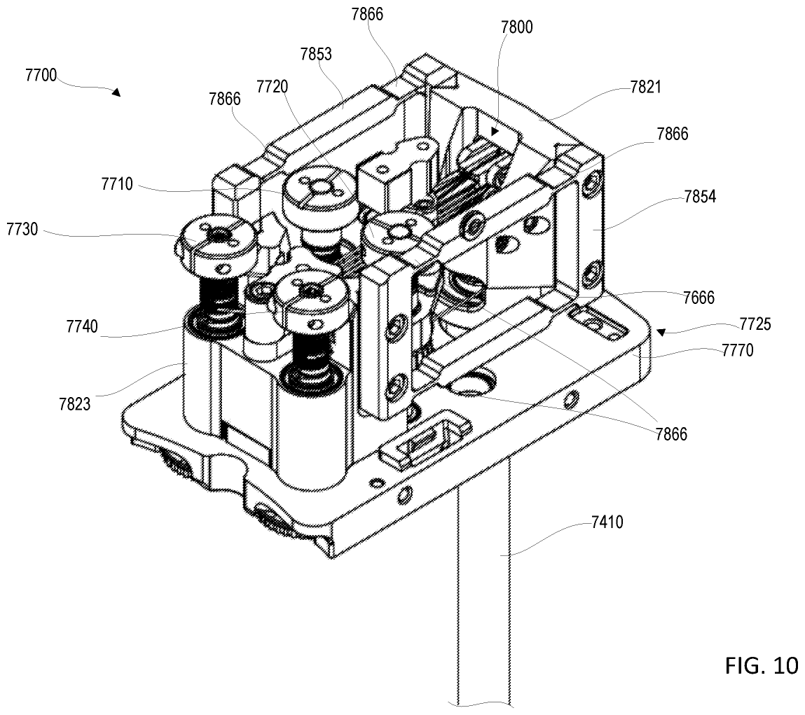

As shown in FIGS. 9 and 10, the mechanical structure 7700 includes four capstans 7510, 7720, 7730 and 7740 (which function as actuator input pieces), and an instrument support structure 7725 that includes a base 7770 and a top plate (not shown), and a circuit board (not shown). In other embodiments, various support structures optionally may be used, such as a chassis, a frame, a bed, a unitized surrounding outer body of the mechanical structure, and the like. The capstans 7710, 7720, 7730, 7740 are motor-driven rollers that rotate or “wind” a portion of the connectors (not shown) to produce the desired connector movement, and therefore the desired movement of the wrist assembly 7500 and end effector 7460. In some embodiments, the mechanical structure 7700 can be constructed the same as or similar to the mechanical structures (referred to as backend assemblies or actuators) or components therein described in U.S. Pat. No. 9,204,923 B2 (filed Jul. 16, 2008), entitled “Medical Instrument Electronically Energized Using Drive Cables,” which is incorporated herein by reference in its entirety.

The shaft translation carriage 7852 movably couples the shaft 7410 relative to the support structure 7725 of the mechanical structure 7700 in a manner such that the amount of shaft movement can be correlated to the applied axial force F_{Z}. The shaft translation carriage 7852 includes components to isolate the axial movement of the shaft 7410 (i.e., to constrain the shaft such that the measured movement is caused only by the axial force F_{Z} and not the transverse forces along the X and Y axes), and limit frictional force opposing movement of the shaft 7410 (which can cause errors in determining the axial force F_{Z}). The shaft translation carriage 7852 includes a resiliency that maintains the shaft 7410 at a defined location along the shaft axis C₁. In other words, there is a lowest energy location along the shaft axis (or along the Z-axis, which can be defined at a reference frame of the support structure 7725) to which the shaft tends, and the shaft translation in a direction away from this lowest energy location causes the resiliency to urge the shaft 7410 back towards the lowest energy location. As a result, the shaft 7410 floats within the outer shaft 7910 at a defined location along the support structure reference frame's Z-axis. The resiliency (i.e., a portion of the shaft translation carriage 7852) is configured to be displaced in proportion to a force imparted to the shaft 7410 in a direction along the shaft axis and the resiliency counteracts the force applied at a distal end of the shaft 7410. Although the resiliency is described below as being produced by a first translation flexure 7853 and a second translation flexure 7854 (which have living hinges 7866), in other embodiments, various spring arrangements can be used to provide the resiliency. For example, some embodiments can include a single spring (not shown) that provides +Z forces and −Z forces, or two springs that each provide +Z or −Z forces.

The amount of travel of the shaft 7410 for a given amount of axial force F_{Z} depends in part on the stiffness of the flexures and living hinges included within medical device 7400 to provide the resiliency as described above. Thus, the shaft translation carriage 7852 is calibrated to provide the desired range of motion of the shaft 7410 over the expected range of axial force. This arrangement can be used to translate (or correlate) the applied axial forces into a displacement that is associated with the axial force F_{Z} applied. Thus, a displacement signal (from the force sensor unit 7800) can be correlated to the applied axial force.

In this embodiment, the shaft translation carriage 7852 includes a first link 7821 (which functions as a shaft roll carrier), a first translation flexure 7853, a second translation flexure 7854 and a second link 7823 coupled together within the mechanical structure 7700. The second link 7823 is fixed to the mechanical structure 7700 (which functions as a ground for the shaft translation carriage 7852). The first link 7821 is configured to constrain shaft translation relative to the support structure of the mechanical structure 7700 to translation along the shaft axis. The first link 7821 is also structured to constrain shaft roll relative to the support structure 7725 of the mechanical structure 7700 to roll about the shaft axis C₁. The two translation flexures 7853 and 7854 provide a resiliency associated with the first link 7821 and translation carriage 7852 that urges the shaft 7410 to a defined lowest energy location along the shaft axis C₁. For example, as shown in FIGS. 9-13, the first flexure 7853 and the second flexure 7854 each include living hinges 7866 on a top portion and a bottom portion of the as shown. The living hinges 7866 are thin portions of material that deform (thereby causing the first flexure 7853 and the second flexure 7854 to deform) when the shaft 7410 (and the first link 7821) move in in the Z-axis direction along the shaft axis C₁.

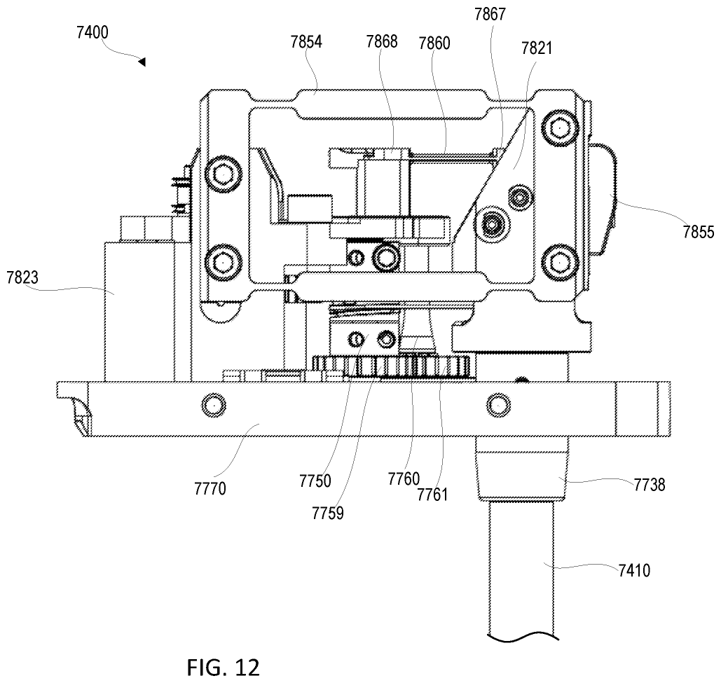

The roll drive group 7744 is configured to rotate the shaft 7410 about the shaft axis C₁ and includes a shaft roll driver 7750, a shaft roll drive receiver 7738 and a shaft roll drive coupling 7746. The shaft roll driver 7750 is coupled to the support structure 7725 of the mechanical structure 7700 and is operatively coupled to the shaft roll drive receiver 7738 via the shaft roll drive coupling 7746 (see, e.g., FIGS. 15 and 16). The shaft roll drive coupling 7746 can be, for example, a cable. The shaft roll driver 7750 is a motor-driven member that produces rotation of the shaft 7410. For example, the shaft roll driver 7750 includes a gear 7751 that engages a gear 7761 of a motor-driven actuation capstan 7760 (see, e.g., FIGS. 12 and 15). When actuated by the actuation capstan 7760, the shaft roll driver 7750 is rotated, which in turn causes the shaft roll drive receiver 7738 to rotate via the shaft roll drive coupling 7746, and rotation of the shaft roll drive receiver 7738 causes rotation of the shaft 7410 relative to the support structure 7725 of the mechanical structure 7700. For example, the shaft roll drive receiver 7738 is actuated by the shaft roll drive coupling 7746 (e.g., a cable, band, cord or other suitable connector coupled to the shaft roll driver 7750 and wound about a portion of the shaft roll drive receiver 7738 as shown, for example, in FIGS. 15 and 16).

The shaft roll drive receiver 7738 is coupled to the shaft 7410 such that when the shaft 7410 translates along the shaft axis C₁ (e.g., Z-axis), the shaft roll drive receiver 7738 also translates along the shaft axis C₁ relative to the shaft roll driver 7750. The shaft roll drive receiver 7738 incudes a bearing 7759 (see, e.g., FIGS. 15 and 16) that engages an inside wall of the first link 7821, such that as the shaft roll drive receiver 7738 translates with the shaft 7410, the first link 7821 also translates along the shaft axis C₁. In other words, the first link 7821 of the shaft translation carriage 7852 is coupled to the shaft roll drive receiver 7738 and to the shaft 7410 such that the first link 7821 can also translate along the shaft axis C₁ with the shaft 7410.

The first link 7821 is structured to constrain translation of the shaft 7410 relative to the support structure of the mechanical structure 7700 to translation along the shaft axis C₁. Said another way, the shaft 7410 is coupled to the first link 7821 in a manner that restricts translational movement of the shaft 7410 relative to the first link 7821 along the Z-axis. Thus, the shaft translation carriage 7852 and the first link 7821 are configured to constrain the shaft 7410 to prevent tilting or “off-axis” movement of the shaft 7410 when it is moved due to the axial force F_{Z}. As described above, the shaft roll driver 7750 is configured to cause the shaft roll drive receiver 7738 to rotate, which in turn causes the shaft 7410 (coupled thereto) to rotate relative to the support structure 7725 of the mechanical structure 7700, but the first link 7821 of the shaft translation carriage 7852 does not rotate with the shaft roll drive receiver 7738 and the shaft 7410. In other words, the first link 7821 of the shaft translation carriage 7852 translates along the shaft axis C₁ with the shaft 7410, but does not rotate with the shaft 7410. This arrangement allows the shaft 7410 to translate along the Z-axis relative to the mechanical structure 7700 (which allows measurement of the axial force) while also allowing the shaft 7410 to be rotated about the Z-axis.

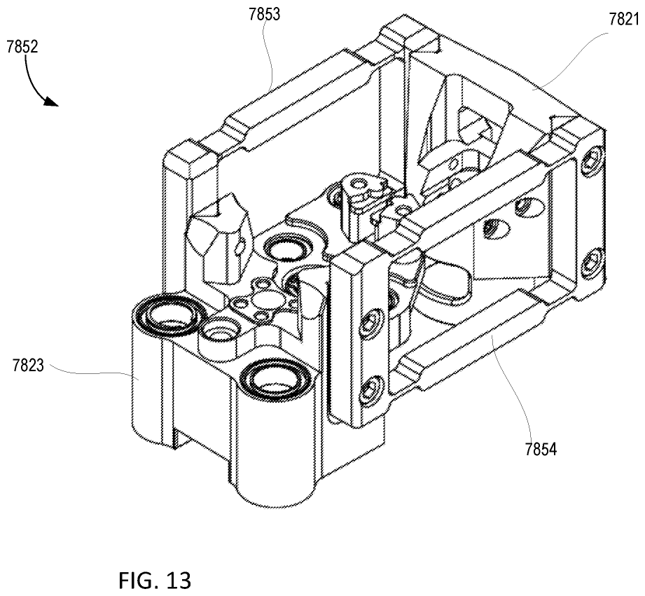

As described above, the resiliency provided by the living hinges 7866 of the first flexure 7853 and the second flexure 7854 can maintain the shaft 7410 at a defined location along the shaft axis. The resiliency is configured to be displaced in proportion to the axial force F_{Z} imparted to the shaft 7410 in a direction along the shaft axis and the resiliency counteracts the axial force F_{Z} applied at a distal end of the shaft 7410. Thus, for example, as the shaft 7410 translates along the Z-axis in a direction toward the mechanical structure 7700, the living hinges 7866 on the first and second flexures 7853 and 7854 allow the flexures 7853 and 7854 to bend or rotate, thus allowing the first link 7821 to translate with the shaft 7410. As shown in FIG. 13, an end portion of each of the flexures 7853 and 7854 is coupled to the second link 7823, and therefore remains stationary with respect to the support structure 7725 of the mechanical structure 7700. The opposite end portion of each of the flexures 7853 and 7854 is coupled to the first link 7821, which moves along the Z-axis. Thus, flexures 7853 and 7854 bend to allow movement of the first link 7821 while the second link 7823 remains fixed. The characteristics of the living hinge (e.g., thickness, material properties) can be adjusted to produce the desired amount of resiliency.

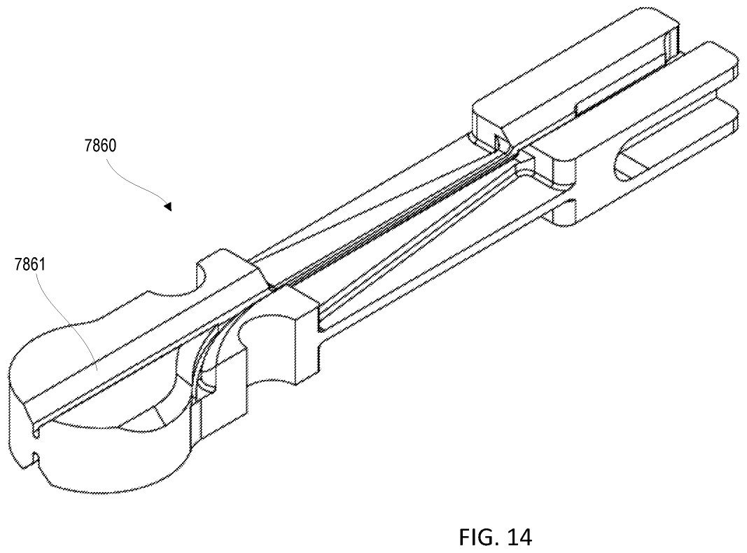

The shaft translation sensor of the force sensor unit 7800 measures the displacement of the shaft 7410 along the Z-axis, which is then converted to a force measurement. In this embodiment, the force sensor unit 7800 includes a shaft translation sensor in the form of an optical fiber sensor (not shown) coupled to a force sensor flexure 7860. The optical fiber sensor can be, for example, a Fiber Bragg Grating (FBG) optical fiber sensor. As shown, for example, in FIG. 14, the force sensor flexure 7860 defines grooves 7861 in which the optical fiber sensor can be disposed. As shown in FIG. 12, a first end 7867 of the force sensor flexure 7860 is coupled to the first link 7821 of the shaft translation carriage 7852 via a slider 7855 and a second end 7868 of the force sensor flexure 7860 is coupled (directly or indirectly) to the stationary second link 7823. Thus, as the shaft 7410 translates along the Z-axis, the first link 7821 translates along the Z-axis, which in turn causes the force sensor flexure 7860 to bend an amount that correlates to the amount of force imparted on the shaft 7410. The optical fiber sensor within the grooves 7861 can accurately measure the magnitude of the deflection of the force sensor flexure 7860 due to changes in the optical signal within the sensor. The amount of deflection is associated with the translation of the shaft 7410 along the Z-axis, and is therefore also associated with the magnitude of the axial force F_{Z} applied. The force sensor unit 7800 can also include a microprocessor that receives the signals from the shaft translation sensor that is associated with a linear displacement of the shaft 7410 along the shaft axis C₁ of the shaft (e.g., along the Z-axis). The microprocessor is configured to execute instructions to determine a measure of a force on the shaft along the shaft axis.

During use of the medical device 7400, as force is imparted on the shaft 7410 in a Z-direction, the shaft 7410 will travel along the Z-axis, which in turn causes the first link 7821 of the shaft translation carriage to translate along the Z-axis. The first end 7867 of the force sensor flexure 7860 and optical fiber sensor are coupled to the first link 7821 such when the shaft 7410 moves axially due to forces imparted on a distal end of the medical device 7400, the force sensor flexure 7860 will deflect or bend an amount corresponding to a distance the shaft 7410 has traveled along the Z-axis. The optical fiber sensor is coupled to the force sensor flexure 7860 such that the amount of bend on the force sensor flexure 7860 is sensed by the optical fiber sensor, which can be translated to a Z-axis force measurement. A microprocessor receives the signals from the optical fiber sensor that is associated with a linear displacement of the shaft along the shaft axis (e.g., along the Z-axis). The microprocessor is configured to execute instructions to determine a measure of a force on the shaft along the shaft axis.

Although shown as including an optical fiber sensor, in other embodiments, the force sensor unit 7800 (or any of the force sensor units described herein) can include any other suitable types of force sensors as described herein. For example, in other embodiments, the force sensor unit 7800 can include various types of strain gauges, including but not limited to conventional foil type resistance gauges, semiconductor gauges, an inductive coil force sensor, an electromagnetic sensor, or an optical sensor (e.g., time-of-flight (TOF)) or others, such as strain sensing surface acoustic wave (SAW) devices. The shaft translation sensor can be coupled to a shaft translation information receiver (not shown) that can receive the shaft translation information (not shown) and can route that information for further processing to produce a haptic sensation force. The shaft translation information receiver can be incorporated into the medical device 7400 or be coupled thereto and can communicate with the force sensor.

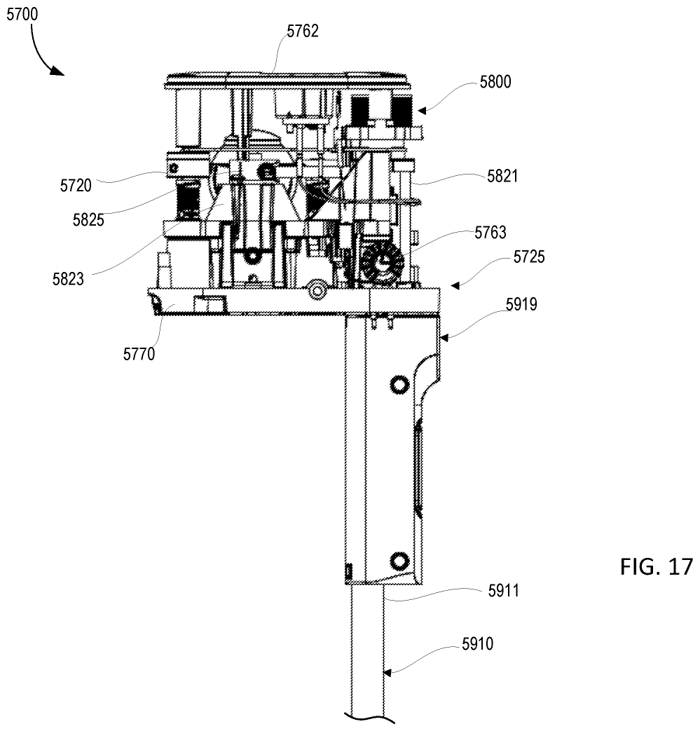

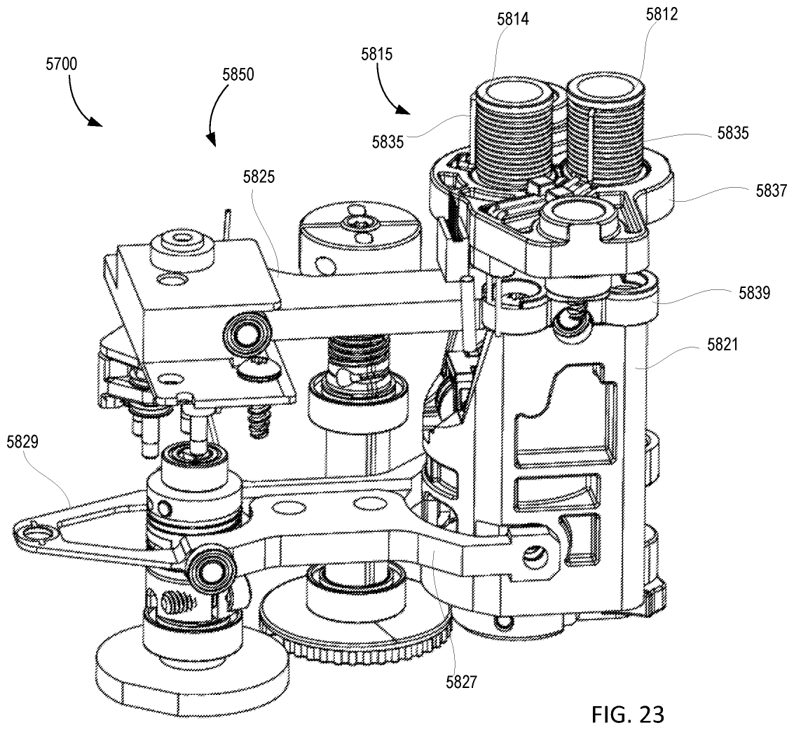

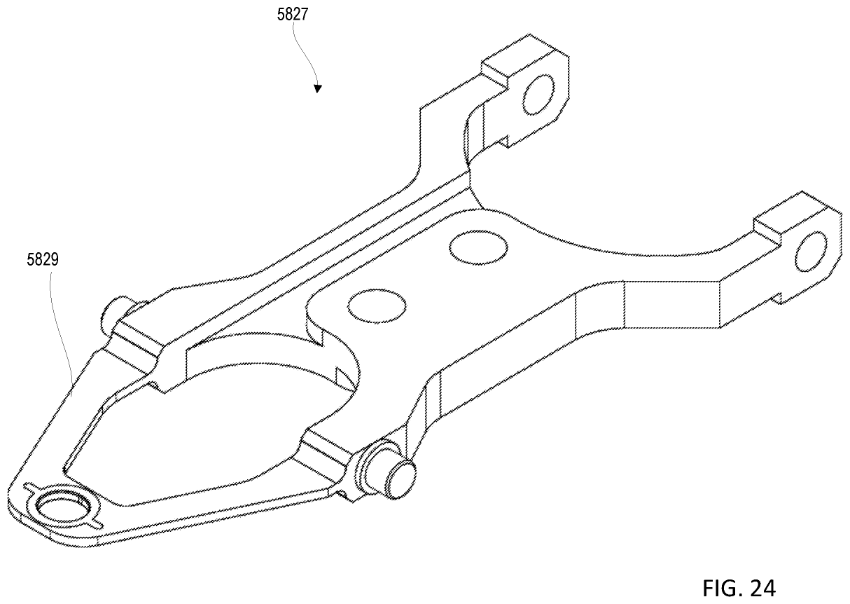

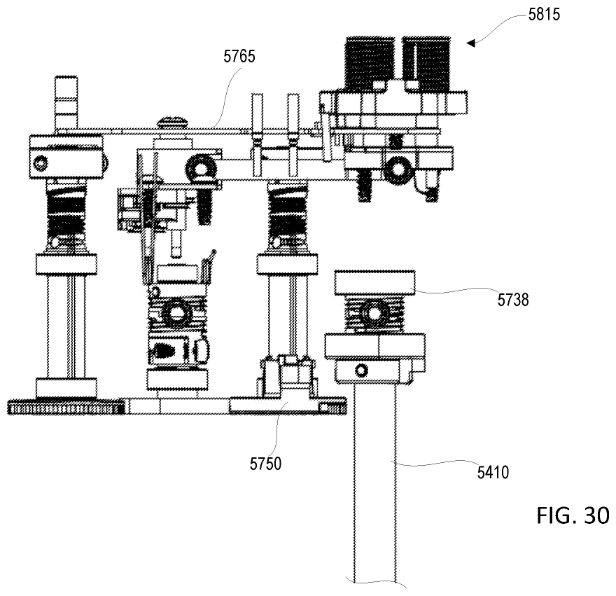

FIGS. 17-30 are various views of a medical device 5400 and its components, according to an embodiment. In some embodiments, the medical device 5400 or any of the components therein are optionally parts of a surgical system that performs surgical procedures, and which can include a manipulator unit, a series of kinematic linkages, a series of cannulas, or the like. The medical device 5400 (and any of the instruments described herein) can be used in any suitable surgical system, such as the MIRS system 1000 shown and described above. The medical device 5400 includes a mechanical structure 5700 at a proximal end portion of the medical device 5400, an outer shaft 5910, a shaft 5410 (which functions as an inner shaft in this embodiment), a force sensor unit 5800, and a distal end mechanism (not shown) that can include a wrist assembly and an end effector as described above for previous embodiments. Although not shown, the instrument 5400 can also include one or more connectors that couple the mechanical structure 5700 to the wrist assembly and end effector, and function as tension members to actuate the end effector. In some embodiments, the connectors can be a cable, a band or the like. The instrument 5400 is configured such that select movements of the connectors produces rotation of the wrist assembly (i.e., pitch rotation) about a first axis of rotation A₁ (see FIG. 8) (which functions as a pitch axis, the term pitch is arbitrary), yaw rotation of the end effector about a second axis of rotation A₂ (see FIG. 8) (which functions as the yaw axis, the term yaw is arbitrary), a cutting rotation of the tool members of the end effector 5460 about the second axis of rotation A₂, or any combination of these movements. Changing the pitch or yaw of the instrument 5400 can be performed by manipulating the connectors in a similar manner as described, for example, in U.S. Pat. No. 8,821,480 B2 (filed Jul. 16, 2008), entitled “Four-Cable Wrist with Solid Surface Cable Channels,” which is incorporated herein by reference in its entirety. Thus, the specific movement of each of the connectors to accomplish the desired motion is not described below.

The shaft 5410 includes a proximal end 5411 that is coupled to the mechanical structure 5700, and a distal end that is coupled to a beam (not shown) in a similar manner as described for medical device 7400. The proximal end of the shaft 5410 is coupled to the mechanical structure 5700 in a manner that allows movement of the shaft 5410 along a shaft axis C₃ (the shaft axis C₃ is analogous to the shaft axis C₁ shown in FIG. 8) relative to the mechanical structure 5700. More specifically, in this embodiment, the shaft 5410 extends through an opening (not sown) in a base 5770 of the mechanical structure 5700 and is coupled to a first link 5821 of a linkage 5850 that allows for the shaft 5410 to translate in the Z-axis direction and also rotate, as described in more detail below. Allowing the shaft 5410 to “float” in the Z direction facilitates measurement of forces along the Z axis, as described herein. The shaft 5410 also defines a lumen (not shown) and/or multiple passageways through which the connectors and other components (e.g., electrical wires, ground wires, or the like) can be routed from the mechanical structure 5700 to the wrist assembly.

The outer shaft 5910 can be any suitable elongated shaft that can be disposed over the shaft 5410 and includes a proximal end 5911 that can be coupled to the mechanical structure 5700 and a distal end (not shown). The outer shaft 5910 defines a lumen between the proximal end 5911 and the distal end. The shaft 5410 extends within the lumen of the outer shaft 5910 and can move relative to the outer shaft 5910. For example, the shaft 5410 can rotate relative to the outer shaft 5910 and/or can translate longitudinally in a direction parallel to the shaft axis C₃ of the shaft 5410 (i.e., the Z-direction). In this embodiment, the proximal end 5911 of the outer shaft 5910 is coupled to a locking handle 5919 that is fixedly coupled to the mechanical structure 5700, as shown in FIG. 17. The locking handle 5919 can be used to move the outer shaft 5910 relative to the shaft 5410 and lock the outer shaft 5910 in a position along the Z-axis direction relative to the shaft 5410. In this manner, the outer shaft 5910 can be retracted (i.e., moved proximally) relative to the shaft 5410 to expose distal portions of the medical device 5400 (e.g., a force sensor beam) to facilitate cleaning of the beam or any sensors coupled thereto. In some embodiments, the locking handle 5919 can be constructed the same as or similar to, and function the same as or similar to the outer shaft mounting tube assembly 970 shown and described in co-pending International Application No. PCT/US2020/055794 (filed Oct. 15, 2020), entitled “Surgical Tool with Nested Shaft Tubes,” the disclosure of which is incorporated herein by reference in its entirety. In other embodiments, the outer shaft 5910 or portions thereof can move relative to the mechanical structure 5700 (e.g., the outer shaft 5910 can be a telescoping shaft).

The mechanical structure 5700 includes components to produce movement of the connectors (not shown) to produce the desired movement (pitch, yaw, or grip) at the wrist assembly (not shown, but which can be similar to the wrist assembly 7500 described herein). Specifically, the mechanical structure 5700 includes components and controls to move some of the connectors in a proximal direction (i.e., to pull in certain connectors) while simultaneously allowing the distal movement (i.e., releasing or “paying out”) of other of the connectors in equal lengths. In this manner, the mechanical structure 5700 can maintain the desired tension within the connectors, and in some embodiments, can ensure that the lengths of the connectors are conserved (i.e., moved in equal amounts) during the entire range of motion of the wrist assembly. In other embodiments, however, conservation of the lengths of the connectors is not required.

In some embodiments, the mechanical structure 5700 can include one or more mechanisms that produce translation (linear motion) of a portion of the connectors. Such a mechanisms can include, for example, a gimbal, a lever, or any other suitable mechanism to directly pull (or release) an end portion of any of the connectors. For example, in some embodiments, the mechanical structure 5700 can include any of the mechanical structures (referred to as backend assemblies or actuators) or components described in U.S. Patent Application Pub. No. US 20157/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

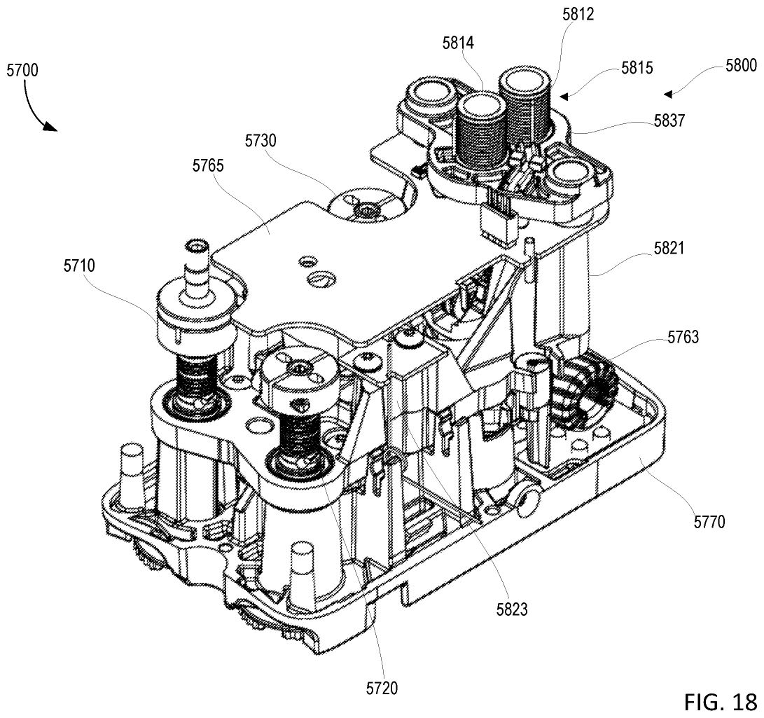

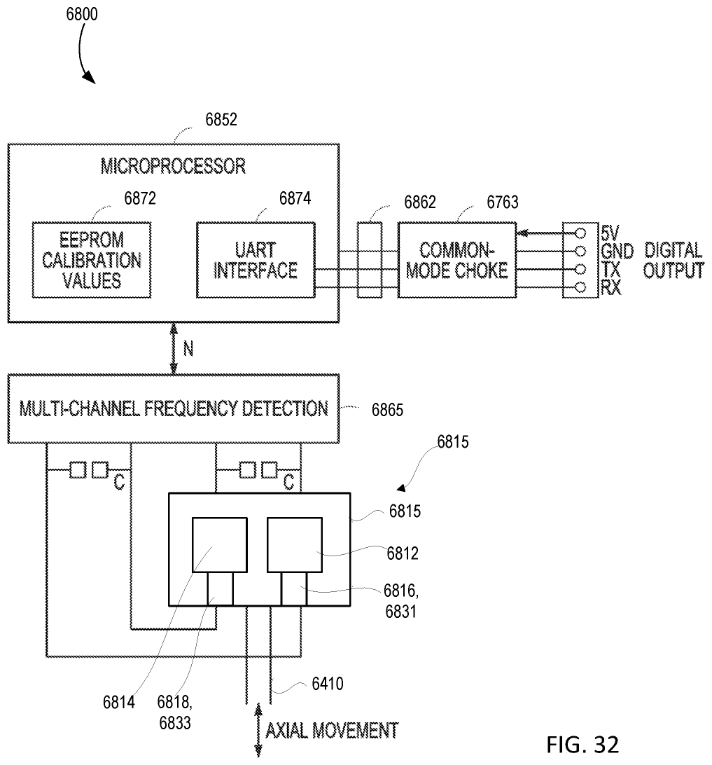

As shown in FIGS. 17-18, the mechanical structure 5700 includes three capstans 5720, 5730 and 5740 (which function as actuator input pieces), and a roll driver 5750, which each function as operation input pieces. The capstans 5720, 5730, 5740 are motor-driven rollers that rotate or “wind” a portion of the connectors (not shown) to produce the desired connector movement, and therefore the desired movement of the wrist assembly and end effector. As described below, the roll driver 5750 is a motor-driven member that produces rotation (referred to as “roll”) of the shaft 5410. In some embodiments, the mechanical structure 5700 can be constructed the same as or similar to the mechanical structures (referred to as backend assemblies or actuators) or components therein described in U.S. Pat. No. 9,204,923 B2 (filed Jul. 16, 2008), entitled “Medical Instrument Electronically Energized Using Drive Cables,” which is incorporated herein by reference in its entirety. The mechanical structure 5700 also includes an instrument support structure 5725 that includes the base 5770 and a top plate 5762, a circuit board 5765 and a common-mode choke 5763 (discussed in more detail below with reference to FIG. 32). In other embodiments, various support structures optionally may be used, such as a chassis, a frame, a bed, a unitized surrounding outer body of the mechanical structure, and the like.

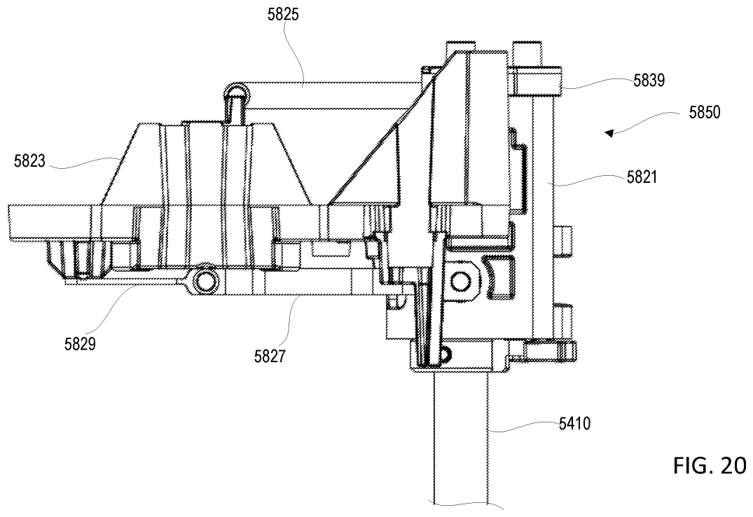

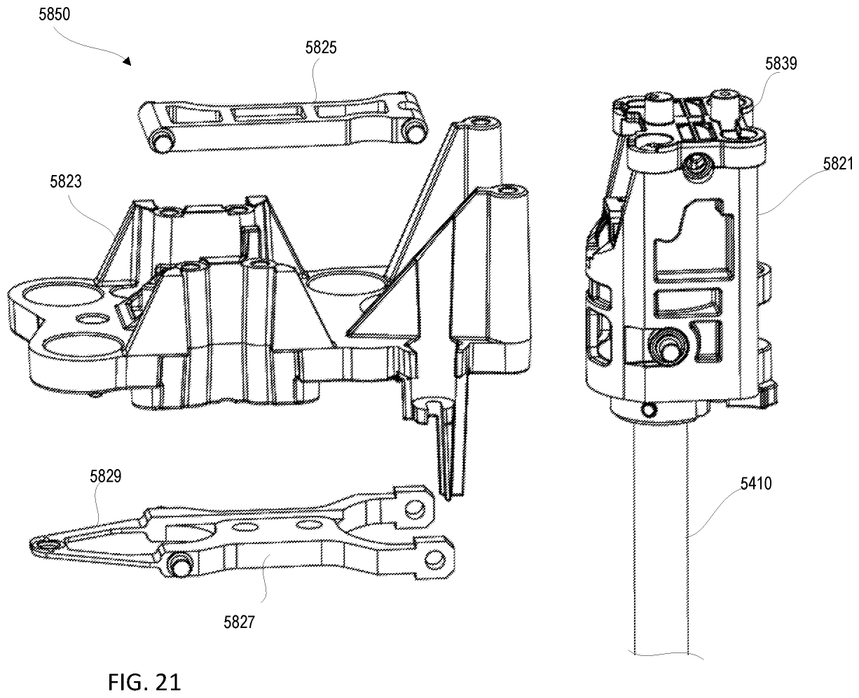

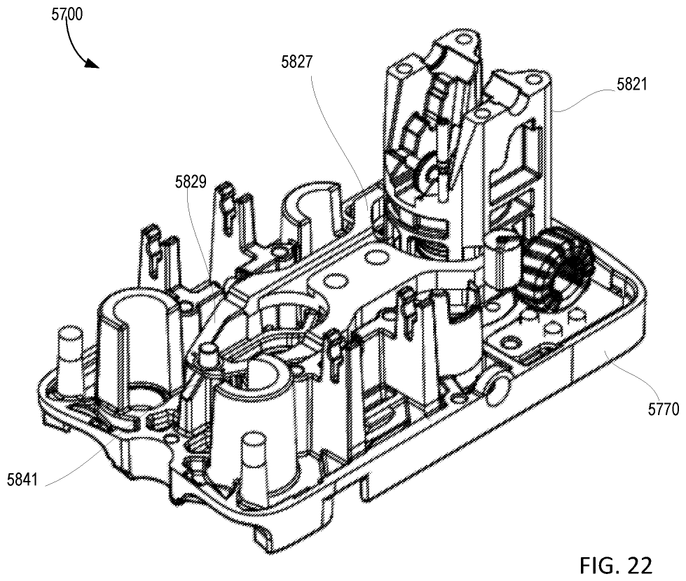

The mechanical structure 5700 surrounds (or is coupled to) the force sensor unit 5800, which includes a coil assembly 5815, a linkage 5850 (which functions as a shaft translation carriage or a movable four-bar linkage) and a microprocessor (see example microprocessor in FIG. 32). The linkage 5850 includes four links coupled to the mechanical structure 5700. More specifically, as best shown in FIGS. 20 and 21, the linkage 5850 includes a first link 5821 (which functions as a roll carrier) coupled to the shaft 5410, a second link 5827 coupled to the first link 5821 and coupled to a support mount 5841 (see, e.g., FIG. 22). A bracket 5839 is coupled to an end of the first link 5821 and is used to couple the rods of the coil assembly 5815 to the first link 5821, as described in more detail below. The second link 5827 includes a spring element 5829 that is coupled to or supported by the support mount 5841, as shown in FIG. 22. The linkage 5850 also includes a third link 5825 coupled to the first link 5821 and a fourth link 5823 coupled to the second link 5827 and the third link 5825. The fourth link 5823 is stationary and acts as a “ground” for the other three links, which move when the shaft 5410 is moved along its axis C-A. The four links of the linkage 5850 can maintain connector tension within the medical device 5400, and provide for linear movement of the shaft 5410 when forces are applied axially at the distal end of the medical device 5400. The linkage 5850 can also constrain the movement in the Z-axis and isolates forces in the Z-axis.

The shaft 5410 is coupled to the mechanical structure 5700 via the linkage 5850 such that the amount of travel of the shaft 5410 relative to the mechanical structure 5700 can be correlated to the magnitude of the axial force imparted to the end effector 5460. In this manner, measuring the distance through which the shaft 5410 moves relative to the mechanical structure 5700 can be used to determine the axial force (e.g., the force in the Z-direction) applied the distal end of the shaft 5410 (e.g., at the end effector). As described herein, the linkage 5850 isolates the axial movement of the shaft 5410 (i.e., constrains the shaft movement such that the measured movement is caused only by the axial force, and not the transverse forces along the X and Y axes), limits frictional force opposing movement of the shaft 5410, and provides suitable structure for the coaxially arranged coils as described below.

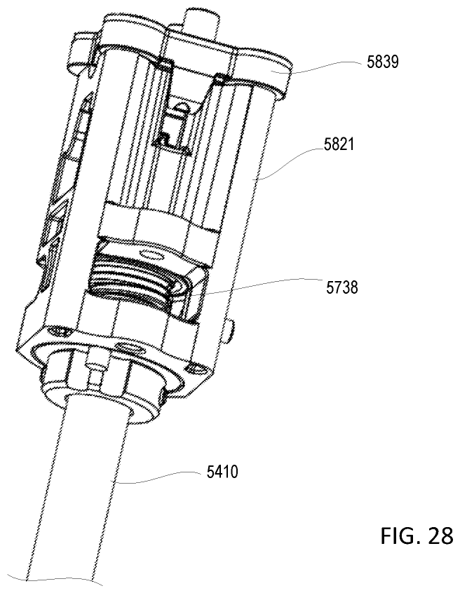

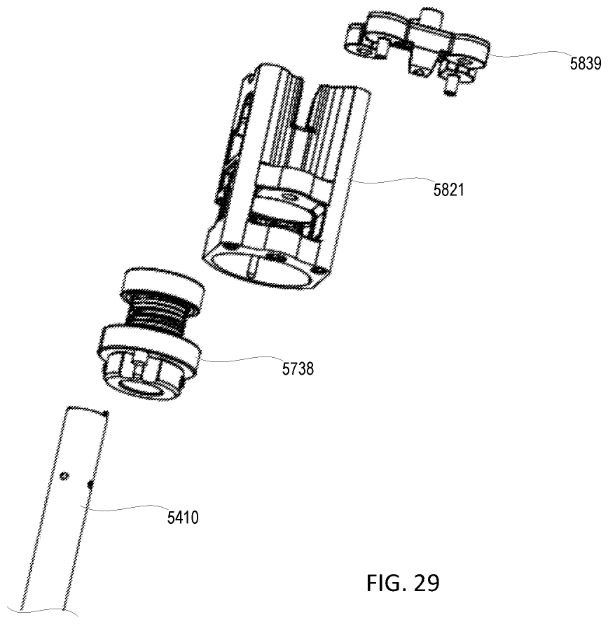

More specifically, the shaft 5410 is coupled to the first link 5821 of the linkage 5850 via a roll receiver 5738 (see, e.g., FIGS. 28-29), such that when the shaft 5410 moves along the Z-axis direction, the first link 5821 moves along the Z-axis direction with the shaft 5410. Said another way, the shaft 5410 is coupled to the first link 5821 in a manner that restricts movement of the shaft 5410 relative to the first link 5821 along the Z-axis. The roll receiver 5738, however, allows for the shaft 5410 to also rotate relative to the first link 5821 (e.g., the first link 5821 does not rotate when the shaft 5410 rotates about the Z-axis). The roll receiver 5738 can be actuated by a cable (band, cord or other suitable connector (not shown)) coupled to a roll driver 5750 and wound about a portion of the roll receiver 5738 as described above for medical device 7400. This arrangement allows the shaft 5410 to move about the Z-axis relative to the mechanical structure 5700 (which allows measurement of the axial force) while also allowing the shaft 5410 to be rotated about the Z-axis.

The spring 5829 of the second link 5827 provides a resiliency associated with the first link 5821 that urges the shaft 5410 to a defined lowest energy location along the shaft axis C₃. Thus, the spring 5829 can maintain the shaft 5410 at a defined location along the shaft axis C₃. In other words, there is a lowest energy location along the shaft axis C₃ (or Z-axis direction relative to the base 5770) to which the shaft tends, and the shaft translation in a direction away from this lowest energy location causes the spring 5829 to urge the shaft 5410 back towards the lowest energy location. As a result, the shaft 5410 floats within the outer shaft 5910 at a defined location along the Z-axis. The spring 5829 is configured to be displaced in proportion to a force imparted to the shaft 5410 in a direction along the shaft axis C₃ and counteracts the force applied at a distal end of the shaft 5410. The spring 5829 can be formed with a material that is more flexible than a remaining portion of the second link 5827, thereby producing a spring with a desired stiffness. The amount of travel of the shaft 5410 in the Z-axis direction depends in part on the stiffness of the spring 5829 of the second link 5827. For example, if the spring 5829 is very stiff, the shaft 5410 will only move a short distance when an axial force is applied to the end effector (not shown). Conversely, if the spring 5829 is less stiff, the same axial force will produce greater movement of the shaft 5410. Thus, the spring 5829 can be selected to have the desired stiffness such that the total travel of the shaft 5410 over the expected range of axial forces to be applied will be within the dynamic range of the force sensor unit 5800. Although the spring 5829 is shown as being a leaf spring, in other embodiments, the linkage 5850 can include any suitable type of spring (e.g., a coil spring or a torsion spring).

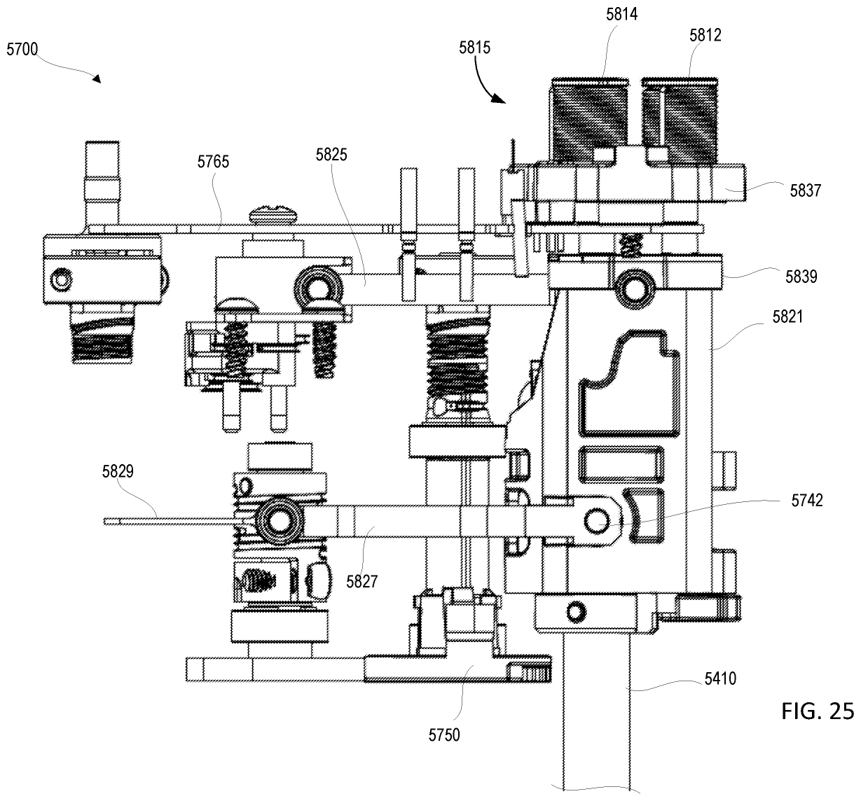

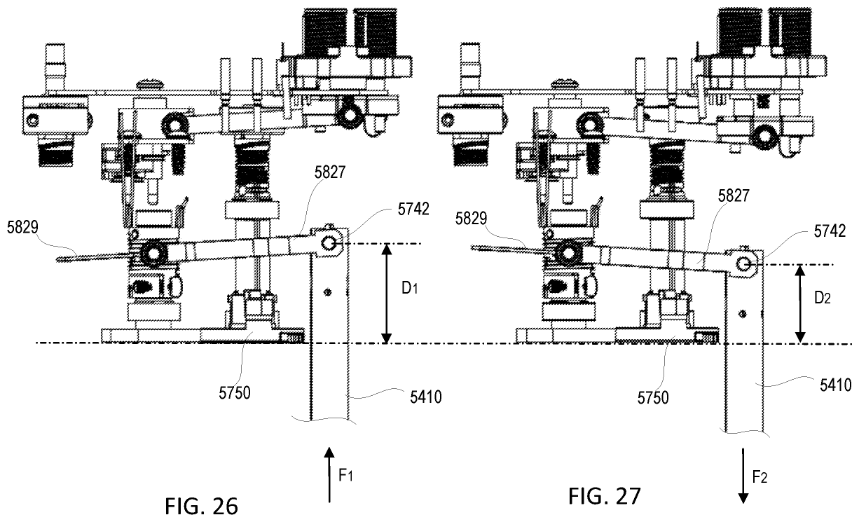

FIG. 25 illustrates a position of the second link 5827 when the second link 5827 and shaft 5410 are in a neutral position (e.g., unactuated). During use of the medical device 5400 when the shaft is moved along the Z-axis direction, the first link 5821 will move with the shaft 5410, and the second link 5827, being coupled to the first link 5821, will pivot about a pivot joint 5742. For example, FIG. 26 illustrates the shaft 5410 translated proximally in the Z-axis direction relative to the neutral position, and FIG. 27 illustrates the shaft 5410 translated distally in the Z-axis direction (the first link 5821 is removed for illustration purposes) relative to the neutral position. The second link 5827 (and spring 5829) is angled downward in FIG. 26 and the pivot joint 5742 is at a distance D₁ from a reference line L. In this configuration (e.g., proximal movement of the shaft 5410), the shaft 5410 is exposed to a first force F₁. The second link 5827 (and spring 5829) is angled upward in FIG. 27 and the pivot joint 5742 is at a distance D₂ from the reference line L, which is less than the distance D₁. In this configuration (e.g., distal movement of the shaft 5410), the shaft is exposed to a second force F₂, which is less than the first force F₁ in this example. By measuring the distance D₁, the magnitude of the first force F1 can be determined, and by measuring the distance D₂ the second force F2 can be determined.

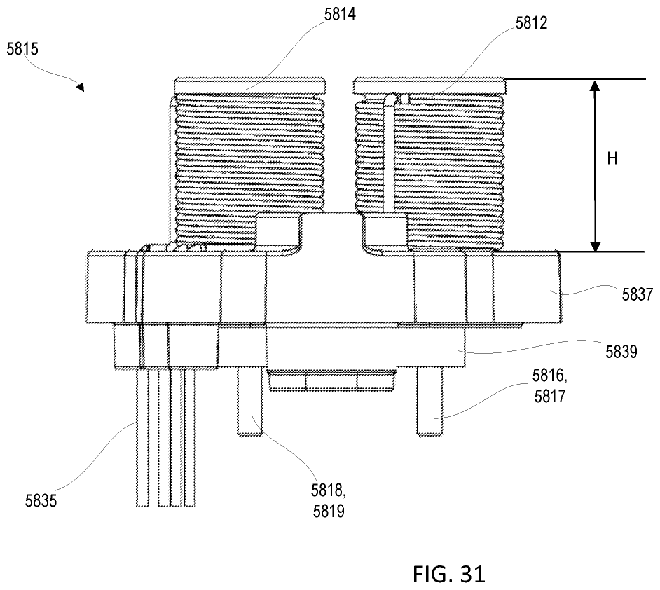

As shown in FIG. 31, the coil assembly 5815 includes a first coil 5812, a second coil 5814, a first rod 5816, a second rod 5818, a first magnet 5831, a second magnet 5833 and a mounting bracket 5837. The mounting bracket 5837 is secured within the mechanical structure 5700 and is electrically coupled to the circuit board 5765 via wiring 5835. The first coil 5812 and the second coil 5814 are each mounted within the mounting bracket 5837 and positioned side-by side to each other. The first coil 5812 and the second coil 5814 are each inductive coils wound around a cylinder formed with a nonconductive material, such as, for example, PEEK. The first coil 5812 and the second coil 5814 are formed with identical characteristics such as coil length (or height), width, and thickness of the coil wire.

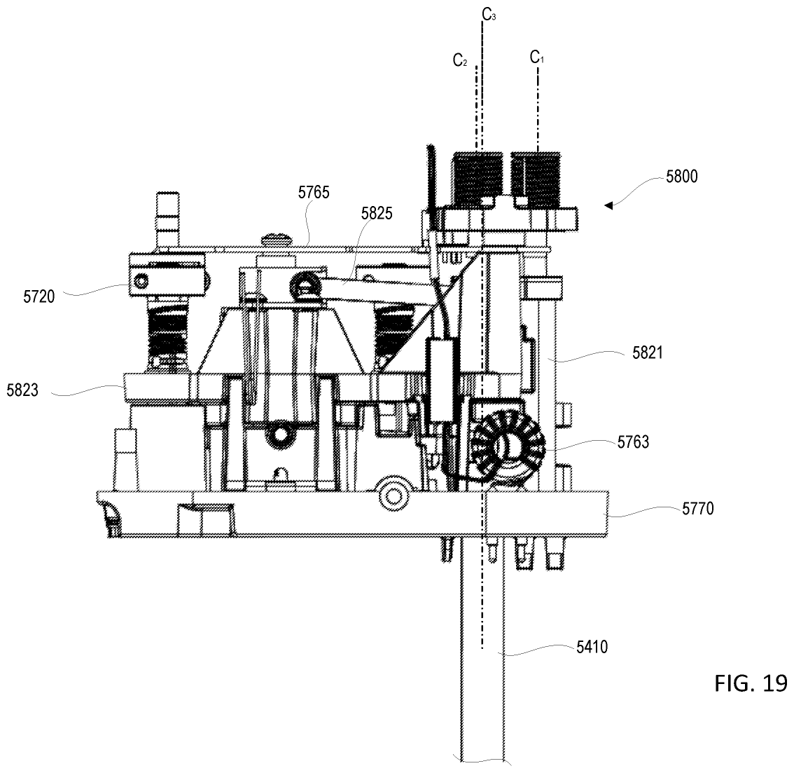

Thus, because the first link 5821 is fixedly coupled to the shaft 5410 in the Z-axis, the first rod 5816 and the second rod 5818 are each fixedly coupled to the shaft 5410 and can move in the Z-axis direction with the shaft 5410 and first link 5821. In some embodiments, the coil assembly 5815 is coupled to the mechanical structure 5700 such that a shaft axis C₃ of the shaft 5410 is between a shaft axis C₁ of the first rod 5816 and a shaft axis C₂ of the second rod 5818 as shown in FIG. 19. In some embodiments, the shaft axis C₃ of the shaft 5410 is parallel to the center axes C₁ and C₂ of the first rod 5816 and the second rod 5816, respectively. In some embodiments, the shaft axis C₃ of the shaft 5410 is centered between the shaft axis C₂ of the first rod 5816 and the shaft axis C₂ of the second rod 5818.

During use of the medical device 5400, as force is imparted on the shaft 5410 in a z-direction, the shaft 5410 will travel along the Z-axis, which in turn causes the rods 5816 and 5816 to translate along the Z-axis (along their respective center axes). As the rods 5816 and 5818 (and magnets 5831 and 5833) move within the respective coils, 5812 and 5814, each of the coils 5812 and 5816 generate a signal associated with a position of the magnets 5831 and 5833 within the respective coil 5812 and 5814. The microprocessor (which can be similar to the microprocessor 6852 shown in FIG. 32) receives these signals from the coils 5812 and 5814. As described above, each of the coils 5812 and 5814 generates a separate signal associated with a linear displacement of the shaft 5410 along the shaft axis C₃ of the shaft 5410 (e.g., along the Z-axis). In some embodiments, the signals from the coils can include a first signal from the first coil 5812 having a first frequency, and a second signal from the second coil 5814 having a second frequency. The microprocessor is configured to execute instructions to determine from the first frequency and the second frequency a measure of a force on the shaft 5410 along the shaft axis C₃ of the shaft 5410.

As described above, the force sensor unit 5800 measures the change in the inductance within the coils due to the Z-axis movement of the shaft (i.e., along the shaft axis C₃ of the shaft 4410), which is converted from position measurement to a force measurement. As described above, the second link 5827 with the spring 5829 and the coil assembly 5815 are grounded to the same rigid component of the mechanical structure 5700 (e.g., the base 5770) such that false force signals due to a difference in deflection in different grounding components can be avoided.

FIG. 32 is a block diagram of a portion of an embodiment of a force sensor unit 6800 that can be implemented to measure axial force applied to an instrument shaft (e.g., shaft 5410). The force sensor unit 6800 can be implemented as an inductive Z-axis force sensor unit as described above for medical device 5400 (including the force sensor unit 5800). As described above, an axial force on the instrument shaft results in an axial movement of the instrument shaft 6410, which can be detected by the force sensor unit 6800. The force sensor unit 6800 can include a coil assembly 6815 as described herein that includes a pair of coils 6812 and 6814 with a rod 6816 and rod 6818 movably positioned within the coils 6812 and 6814, respectively. The rod 6816 can have a magnet 6831 coupled thereto and the rod 6818 can have a magnet 6833 coupled thereto.

The coil 6812 can be coupled to a multi-channel frequency detection 6865 by a capacitor C that can form an inductor/capacitor (LC) circuit with the coil 6812 with an inductance contribution based on the distance the rod 6816 and magnet 6831 move within the coil 6812. The coil 6814 can be coupled to the multi-channel frequency detection 6865 by a capacitor C that can form a LC circuit with the coil 6814 with an inductance contribution based on the distance the rod 6818 and magnet 6833 move within the coil 6814. The LC circuits associated with the coils 6812 and 6814 can be implemented with different capacitances, where such differences are taken into account.

The multi-channel frequency detection 6865 can be implemented as a precision, dual inductance sensor that measures the inductance. With the capacitor C forming an LC circuit with the coil 6812 input to the multi-channel frequency detection 6865, the multi-channel frequency detection 6865 can output a first signal associated with a frequency of this circuit, for example a ratio of the frequency with a known reference frequency. With the capacitor C forming an LC circuit with the coil 6814 input to the multi-channel frequency detection 6865, the multi-channel frequency detection 6865 can output a second signal associated with a frequency of this circuit, for example a ratio of the frequency with a known reference frequency. The multi-channel frequency detection 6865 can output N digital signals to a microprocessor 6852. For two LC circuits, the multi-channel frequency detection 6865 can output two digital signals to the microprocessor 6870.

The microprocessor 6852 can include or have access to an EEPROM 6872, or other storage device, that can include calibration values for implementation of the magnet 6831 within the coil 6812 and the magnet 6833 within the coil 6814. In a measurement of axial force on the instrument shaft, the calibration values can be accessed to determine a distance moved for each magnet 6831 and 6833 based on the frequencies received from the multi-channel frequency detection 6865. The difference in frequencies can be stored in the EEPROM 6872 as a difference of inductance as a function of distances. This difference of distances can be correlated with a reference position and the difference in inductances. With a distance selected from a measured difference in inductances, the distance can be used with a spring constant stored in the EEPROM 6872, where the spring constant is a property of a spring (e.g., spring 5829 described above) by which the instrument shaft 6410 is coupled to a support structure on which the force sensor unit 6800 can be deployed.

The force sensor unit 6800 can include other components. For example, the microprocessor 6852 can include a Universal Asynchronous Receiver/Transmitter (UART) interface 6874 or other communication interface to transmit (TX) a digital output and receive (RX) a digital signal. The received signal can be used to update calibration values in the EEPROM 6872 of the microprocessor 6852. A common-mode choke 6763 (such as common-mode choke 5863) can be used to reduce interference with other boards of the support structure on which the force sensor unit 6800 is deployed. Optionally, the force sensor unit 6800 can include a magnetic structure 6862 between the common-mode choke 6763 and the microprocessor 6852. The magnetic structure 6862 can be inserted to help with electromagnetic interference (EMI) radiation reduction. The magnetic structure 6862 can be realized as a ferrite bead. Other magnetic material formats can be implemented for the magnetic structure 6862.

A machine-readable storage device may include any non-transitory mechanism for storing information in a form readable by a machine, for example, a computer or a microprocessor tasked to perform specific functions. For example, a machine-readable storage device may include read-only memory (ROM), random-access memory (RAM), magnetic disk storage media, optical storage media, flash-memory devices, and other storage devices and media. In various embodiments of a medical device with a force sensor unit described herein, a non-transitory machine-readable medium can comprise instructions, which when executed by a set of processors, can cause a system to perform operations comprising: receiving a first signal generated by a first coil associated with a position of a first magnet with reference to the first coil, and a second signal generated by a second coil associated with a position of a second magnet with reference to a second coil; and where the first signal from the first coil and the second signal from the second coil are associated with a linear displacement of the shaft along the shaft axis. The force sensor unit can comprise a microprocessor coupled to receive the first and second signals. In various embodiments, a non-transitory machine-readable medium can comprise instructions, which when executed by a set of processors cause a system to perform operations comprising methods of performing functions associated with the various embodiments described herein.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, connectors, cables, or other components described herein can be constructed from multiple pieces that are later joined together. For example, in some embodiments, a link can be constructed by joining together separately constructed components. In other embodiments however, any of the links, tool members, beams, shafts, connectors, cables, or components described herein can be monolithically constructed.

Although the instruments are generally shown as having an axis of rotation of the tool members (e.g., axis A₂) that is normal to an axis of rotation of the wrist member (e.g., axis A₁), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A surgical instrument, comprising:
a support structure, a shaft, a shaft translation carriage including a shaft roll carrier, a shaft roll drive group, and a force sensor unit;
wherein the shaft comprises a proximal end and a distal end, and a shaft axis is defined by the proximal and distal ends;
wherein the shaft is coupled to the support structure by the shaft roll carrier;
wherein the shaft roll drive group is configured to rotate the shaft about the shaft axis and comprises a shaft roll driver coupled to the support structure, a shaft roll drive receiver coupled to the shaft, and a shaft roll drive coupling coupled between the shaft roll driver and the shaft roll drive receiver, so that the shaft roll drive receiver translates along the shaft axis relative to the shaft roll driver as the shaft translates along the shaft axis; and
wherein the force sensor unit is configured to produce a signal associated with an amount of a force imparted to the shaft along the shaft axis.

2. The surgical instrument of claim 1, wherein:
the shaft translation carriage is structured to constrain shaft translation relative to the support structure to translation along the shaft axis; and
the shaft roll carrier is structured to constrain shaft roll relative to the support structure to roll about the shaft axis.

3. The surgical instrument of claim 2, wherein:
the shaft translation carriage comprises a resiliency that urges the shaft to a defined lowest energy location along the shaft axis.

4. The surgical instrument of claim 3, wherein:
the resiliency comprises one or more springs coupled between the support structure and the shaft translation carriage.

5. The surgical instrument of claim 3, wherein:
the resiliency is inherent in the shaft translation carriage.

6. The surgical instrument of claim 2, wherein:
the shaft translation carriage comprises a spring; and
the spring is configured to be displaced in proportion to a force imparted to the shaft in a direction along the shaft axis.

7. The surgical instrument of claim 2, wherein:
the force sensor unit further comprises a sensor; and
a signal generated by the force sensor is associated with a linear displacement of the shaft as the shaft translates along the shaft axis.

8. The surgical instrument of claim 2, wherein:
the force sensor unit comprises an inductive sensor and a microprocessor;
the inductive sensor is configured to generate a signal associated with a position of the shaft as the shaft translates along the shaft axis;
the microprocessor is configured to receive the signal and to provide an output based on the signal; and
the output of the microprocessor is associated with a force on the shaft along the shaft axis.

9. The surgical instrument of claim 2, wherein:
the shaft roll drive coupling comprises a cable.

10-19. (canceled)

20. A surgical instrument, comprising:
a mechanical structure, a shaft comprising a proximal end portion and a distal end portion, a force sensor unit, a shaft roll drive receiver coupled to the proximal end portion of the shaft, and a shaft roll driver coupled to the shaft roll drive receiver;
wherein a shaft axis extends between the proximal and distal end portions of the shaft;
wherein the force sensor unit is configured to produce a signal associated with a force imparted to the shaft in a direction along the shaft axis;
wherein the shaft roll drive receiver and the shaft translate along the shaft axis; and
wherein the shaft roll driver rotates the shaft roll drive receiver, and the shaft roll drive.

21. The surgical instrument of claim 20, wherein:
the shaft roll driver comprises a shaft roll drive coupling coupled to the shaft roll drive receiver; and
the shaft roll drive coupling comprises a cable.

22. The surgical instrument of claim 20, wherein:
the surgical instrument further comprises a shaft roll drive carrier coupled to the mechanical structure and to the shaft;
the shaft roll drive carrier is movable with the shaft along the shaft axis; and
the shaft roll drive carrier remains stationary as the shaft rotates about the shaft axis.

23. The surgical instrument of claim 22, wherein:
the surgical instrument further comprises a shaft translation carriage;
the shaft translation carriage comprises a spring; and
the spring is configured to be displaced in proportion to the force imparted to the shaft in the direction along the shaft axis.

24. The surgical instrument of claim 20, wherein:
the force sensor unit further comprises a sensor; and
a signal generated by the sensor is associated with a linear displacement of the shaft as the shaft translates along the shaft axis.

25. The surgical instrument of claim 20, wherein:
the force sensor unit comprises an inductive sensor and a microprocessor communicatively coupled to the inductive sensor;
the inductive sensor is configured to generate a signal associated with a position of the shaft as the shaft moves along the shaft axis;
the microprocessor receives the signal and provides an output based on the signal; and
the output of the microprocessor is associated with a force on the shaft along the shaft axis.

26. The surgical instrument of claim 1, wherein:
the shaft translation carriage comprises a resiliency that urges the shaft to a defined lowest energy location along the shaft axis.

27. The surgical instrument of claim 26, wherein:
the resiliency comprises one or more springs coupled between the support structure and the shaft translation carriage.

28. The surgical instrument of claim 26, wherein:
the resiliency is inherent in the shaft translation carriage.

29. The surgical instrument of claim 1, wherein:
the shaft translation carriage comprises a spring; and
the spring is configured to be displaced in proportion to a force imparted to the shaft in a direction along the shaft axis.

30. The surgical instrument of claim 1, wherein:
the force sensor unit further comprises a sensor; and
a signal generated by the sensor is associated with a linear displacement of the shaft as the shaft translates along the shaft axis.

31. The surgical instrument of claim 1, wherein:
the force sensor unit comprises an inductive sensor and a microprocessor;
the inductive sensor is configured to generate a signal associated with a position of the shaft as the shaft translates along the shaft axis;
the microprocessor is configured to receive the signal and to provide an output based on the signal; and
the output of the microprocessor is associated with the position of the shaft as the shaft translates along the shaft axis.

32. The surgical instrument of claim 1, wherein:
the shaft roll drive coupling comprises a cable.

33. The surgical instrument of claim 20, wherein:
the surgical instrument further comprises a shaft translation carriage;
the shaft translation carriage comprises a spring; and
the spring is configured to be displaced in proportion to the force imparted to the shaft in the direction along the shaft axis.

34. The surgical instrument of claim 21, wherein:
the surgical instrument further comprises a shaft translation carriage;
the shaft translation carriage comprises a spring; and
the spring is configured to be displaced in proportion to the force imparted to the shaft in the direction along the shaft axis.
